(19) United States
(12) Patent Application Publication
(10) Pub. No.: US 2015/0157575 A1
(43) Pub. Date: Jun. 11, 2015

(54) Pharmaceutical Formulations Comprising Vilazodone

(71) Applicant: Forest Laboratories Holdings Ltd., Hamilton (BM)

(72) Inventor: Yan Yang, Roslyn Heights, NY (US)

(21) Appl. No.: 14/565,842

(22) Filed: Dec. 10, 2014

Related U.S. Application Data
(60) Provisional application No. 61/904,107, filed on Dec. 10, 2013.

(51) Int. Cl. A61K 9/20; A61K 31/496

(52) U.S. Cl. CPC A61K 9/2095; A61K 31/496; A61K 9/2059; A61K 9/2054; A61K 9/2018; A61K 9/205; A61K 9/2009; A61K 9/2027

(57) ABSTRACT

The present invention provides an immediate release oral dosage form comprising therapeutically effective amount of vilazodone or a salt thereof and at least one excipient. The dosage form includes 10 to 40 mg of vilazodone or a salt thereof, and is compressed in a tablet formulation. The tablet comprising 40 mg vilazodone or a salt thereof has a hardness of more than 9 kp and less than or equal to 14 kp. The tablet comprising 20 mg vilazodone or a salt thereof has a hardness of more than 8 kp and less than or equal to 12 kp, and the tablet comprising 10 mg vilazodone or a salt thereof has a hardness of more than 6 kp and less than or equal to 9 kp.

### FIELD OF THE INVENTION

The present invention is directed to novel vilazodone tablet formulations comprising vilazodone.

### BACKGROUND OF THE INVENTION

Vilazodone, 1-[4-(5-cyanoindol-3-yl)butyl]-4-(2-carbamoyl-benzofuran-5-yl)-piperazine hydrochloride, is a compound that belongs to the group of indole alkylamines. Vilazodone is a dual-acting serotonergic agent that combines antidepressant effects of selective serotonin-reuptake inhibitors (SSRI) with partial serotonin (5-HT)(1A)-receptor agonist activity. Mechanism of action of the antidepressant effect of vilazodone is thought to be related to its enhancement of serotonergic activity in the central nervous system through selective inhibition of serotonin reuptake. Vilazodone binds with high affinity to serotonin reuptake site but not to norepinephrine or dopamine reuptake site. As a result, vilazodone potently and selectively inhibits the reuptake of serotonin.

Depressive disorders affect approximately 121 million people worldwide, including almost 15 million American adults. Patients with depressive disorders present with, without limiting, depressed mood, loss of interest or pleasure, feelings of guilt or low self-worth, disturbed sleep or appetite, low energy and poor concentration. Vilazodone offers a novel combination of selective serotonin reuptake inhibition and serotonergic receptor partial receptor activity. Because of these characteristics, vilazodone is termed a serotonin partial agonist reuptake inhibitor.

Vilazodone may be used for the treatment and prevention of depressive disorders, anxiety disorders, bipolar disorders, mania, dementia, substance-related disorders, sexual dysfunctions, eating disorders, obesity, fibromyalgia, sleeping disorders, psychiatric disorders, cerebral infract, tension, for the therapy of side-effects in the treatment of hypertension, cerebral disorders, chronic pain, acromegaly, hypogonadism, secondary amenorrhea, premenstrual syndrome, and undesired puerperal lactation.

Vilazodone, polymorphic forms of vilazodone and methods for synthesis of vilazodone are disclosed in U.S. Pat. Nos. 5,532,241, 5,723,614, 5,977,112, 6,531,503, 6,509,475, 6,762,300, 6,900,212, 7,371,756, 7,381,726, 7,479,492, 7,642,261, 7,799,916, 7,834,020, 7,981,894, 8,193,195, 8,236,804, 8,318,744 and in U.S. Patent Application Publication 2013/01022616, disclosures of all of the above U.S. patents and patent application are hereby incorporated by reference in their entirety.

Vilazodone was approved by the FDA for the treatment of Major Depressive Disorder (MDD) in January 2011. Vilazodone (commercially known as Viibryd®) is presently marketed as immediate-release tablets. Current guidelines for use of vilazodone in treatment of major depressive disorder recommends that vilazodone be administered at a starting dose of 10 mg once daily for seven days, followed by 20 mg once daily for additional seven days and then increased to 40 mg once daily.

Granulation of active ingredients is a commonly used technique in the art to prepare dosage forms with optimized hardness and compression force. However, merely granulating active ingredients is insufficient to produce dosage forms having optimized physical properties. Compression force used to prepare tablets play an important role in the physical properties of the dosage forms. Physical properties of the final tablet product such as hardness and dissolution are very important. If the tablet is too hard, it may be too brittle and may break into pieces. Additionally, as tablets become harder, frigility lowers and dispersion/dissolution becomes slower, thus resulting in slow disintegration of the tablet.

Vilazodone tablets are manufactured by a direct compression process. During manufacturing of Vilazodone HCl tablets, a higher than desirable level of tablet breakage was observed. Breakage of tablets comprising vilazodone HCl may result in inadequate drug concentrations and thus may diminish the drug's effectiveness. According to the FDA approved dose regimen, vilazodone must be taken with food. If vilazodone is taken without food, inadequate drug concentrations may result. Hence, there exists a need to control breakage observed of vilazodone HCl tablets as well as improving efficacy in light of the observed food effect.

Attempts have been made to provide immediate-release vilazodone HCl tablets with improved breakage parameters and hardness. Controlling breakage of vilazodone HCl tablets has been technologically demanding, thus requiring a unique method of making such tablets.

### SUMMARY OF THE INVENTION

The present invention provides an immediate release oral dosage form comprising therapeutically effective amount of vilazodone or a salt thereof and at least one excipient. The dosage form comprises 10 to 40 mg of vilazodone or a salt thereof, and is compressed in a tablet formulation. The tablet comprising 40 mg vilazodone or a salt thereof has a hardness of more than 9 kp and less than or equal to 14 kp. The tablet comprising 20 mg vilazodone or a salt thereof has a hardness of more than 8 kp and less than or equal to 12 kp, and the tablet comprising 10 mg vilazodone or a salt thereof has a hardness of more than 6 kp and less than or equal to 9 kp.

The oral dosage form is of thickness of about 0.095-0.195″ and friability is not more than about 1.0%. The tablet has a disintegration time of not more than about 2 minutes and a compression force of about 4 kN to 18 kN. Vilazodone or a salt thereof is present in amounts ranging from about 0.05% w/w to about 50% w/w.

The present invention further provides an immediate release oral dosage form comprising the excipient, which includes one or more diluents, disintegration aids, glidants, lubricants, coloring agents, and opacifying agents. The diluent is selected from the group consisting of lactose, sucrose, glucose, dextrose, microcrystalline cellulose, dibasic calcium phosphate, calcium sulphate, mannitol, erythritol, lactilol, maltitol, xylitol, sorbitol, starch, and mixtures thereof. The diluent is present in amounts ranging from about 20% w/w to about 80% w/w.

The glidant is selected from the group consisting of water soluble excipient, hydrophilic polymers, silicon dioxide, colloidal silicon dioxide, microcrystalline cellulose, and combinations thereof. The glidant is present in amounts ranging from about 0.05% w/w to about 5% w/w.

The disintegration aid is selected from the group consisting of ion exchange resin, hydroxypropylcellulose, crospovidone, croscarmellose sodium, starches, pectins, alginates, surfactants, microcrystalline cellulose, sodium starch glycolate, and combinations thereof. The disintegration aid is present in amounts ranging from about 5% w/w to about 30% w/w.

The lubricant is selected from the group consisting of magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, zinc stearate, and combinations thereof. The lubricant is present in amounts ranging from about 0.3% w/w to about 10% w/w.

The present invention further provides an immediate release oral dosage form comprising a therapeutically effective amount of vilazodone or a salt thereof, and at least one excipient, wherein the dosage form comprises 20 mg vilazodone or a salt thereof, having hardness of more than 8 kp and less than or equal to 12 kp, and produces in subjects with severe hepatic impairment an effect comprising at least one of: a mean Cₘₐₓ of about 22 ng/ml or more; a mean Tₘₐₓ of about 3 hours or more; or a mean AUC_{0-∞} of more than about 550 ng h/ml. The mean Cₘₐₓ in subjects with severe hepatic impairment is about 25% lower than mean Cₘₐₓ of subjects without severe hepatic impairment. The present invention further provides an immediate release oral dosage form comprising pharmacokinetic profile as shown in FIG. 8.

The present invention provides an immediate release oral dosage form comprising a therapeutically effective amount of vilazodone or a salt thereof, and at least one excipient, wherein the dosage form comprises 20 mg vilazodone or a salt thereof, having hardness of more than 8 kp and less than or equal to 12 kp, and produces in subjects with moderate hepatic impairment an effect comprising at least one of a mean Cₘₐₓ of about 22 ng/ml or more; a mean Tₘₐₓ of about 2 hours or more; or a mean AUC_{0-∞} of more than about 450 ng h/ml. The present invention further provides an immediate release form comprising pharmacokinetic profile as shown in FIG. 7.

The present invention further provides an immediate release oral dosage form comprising a therapeutically effective amount of vilazodone or a salt thereof, and at least one excipient, wherein the dosage form comprises 20 mg vilazodone or a salt thereof, having hardness of more than 8 kp and less than or equal to 12 kp, and produces in subjects with mild hepatic impairment produces in the subjects an effect comprising at least one of a mean Cₘₐₓ of about 14 ng/ml or more; a mean Tₘₐₓ of about 3 hours or more; or a mean AUC_{0-∞} of more than about 300 ng h/ml. The immediate release oral dosage form comprises pharmacokinetic profile as shown in FIG. 7.

The present invention further provides an immediate release oral dosage form, wherein the dosage form comprises 20 mg vilazodone or a salt thereof, having hardness of more than 8 kp and less than or equal to 12 kp, and produces in the subjects with severe hepatic impairment a concentration of metabolite M17 of vilazodone or a salt thereof comprising at least one of: a mean Cₘₐₓ of about 1 ng/ml or more; a mean Tₘₐₓ of about 3 hours or more; or a mean AUC_{0-∞} of more than about 20 ng h/ml. The immediate release oral dosage form produces in subjects with severe hepatic impairment a concentration of metabolite M17 of vilazodone or a salt thereof, that is at least 42% lower in subjects with severe impairment than the concentration of M17 in subjects without severe hepatic impairment.

The present invention further provides an immediate release oral dosage form, wherein the dosage form comprises 40 mg of vilazodone or a salt thereof, wherein the dosage form comprises 20 mg vilazodone or a salt thereof, having hardness of more than 8 kp and less than or equal to 12 kp, and produces an in vivo plasma profile of metabolite M17 of vilazodone or a salt thereof, comprising at least one of: a mean Cₘₐₓ of about 5 ng/ml or more; a mean Tₘₐₓ of about 5 hours or more; or a mean AUC₀₋₂₄ of more than about 80 ng h/ml.

The present invention further provides an immediate release oral dosage form comprising vilazodone or a salt thereof, wherein the dosage form of 40 mg of vilazodone or a salt thereof produces an in vivo plasma profile comprising at least one of: a mean Tₘₐₓ of about 4 hours or more; a mean Cₘₐₓ of less than about 180 ng/ml; or a mean AUC₀₋₂₄ of less than about 1800 ng h/ml.

The present invention further provides a method of treating a subject with severe hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising administering to said subject the oral dosage form comprising a therapeutically effective amount of 20 mg vilazodone or a salt thereof, having hardness of more than 8 kp and less than or equal to 12 kp.

The present invention further provides a method of treating a subject with moderate hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising administering to said subject the oral dosage form comprising a therapeutically effective amount of 20 mg vilazodone or a salt thereof, having hardness of more than 8 kp and less than or equal to 12 kp.

The present invention further provides a method of treating a subject with mild hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising a therapeutically effective amount of 20 mg vilazodone or a salt thereof, having hardness of more than 8 kp and less than or equal to 12 kp.

### BRIEF DESCRIPTION OF THE FIGURES

FIG. 1 shows the process of pre-blending active ingredients.

FIG. 2 shows the process of pre-blending excipients.

FIG. 3 shows the process of fmal blending.

FIG. 4 is a graph comparing the dissolution profiles of 10 mg vilazodone HCl tablets.

FIG. 5 is a graph comparing the dissolution profiles of 20 mg vilazodone HCl tablets.

FIG. 6 is a graph comparing the dissolution profiles of 40 mg vilazodone HCl tablets.

FIG. 7 shows mean plasma vilazodone concentration-time profiles of participants with mild or moderate hepatic impairment and their matched healthy controls.

FIG. 8 shows mean plasma vilazodone concentration-time profiles of participants with severe hepatic impairment and their matched healthy controls.

FIG. 9 is a schematic showing a study design to evaluate the effect of steady-state carbamazepine XR on the pharmacokinetics of steady-state vilazodone.

FIG. 10 shows plasma concentration-time profiles for vilazodone and M17 following vilazodone alone and vilazodone with carbamazepine XR administration.

FIG. 11 shows the mean plasma concentration-time profiles for carbamazepine and carbamazepine-10,11-epoxide following multiple-dose carbamazepine XR alone and with vilazodone at steady state treatment.

### DEFINITIONS

For purposes of the present invention, the term “immediate release” as used in the present invention takes its art-recognized meaning. A drug formulation is considered to be “immediate release” if it meets disintegration and/or dissolution requirements for immediate release solid oral dosage forms as set out, for example, in the United States Pharmacopoeia (USP).

The term “dissolution requirement” means the dissolution rate of vilazodone obtained when tested using the equipment and procedure specified in the USP and conducted pursuant to the individual Official Monographs of USP for the particular therapeutically active agent(s).

A “therapeutically effective amount” means the amount of a compound that, when administered to a mammal for treating a state, disorder or condition is sufficient to effect a treatment (as defined below). The “therapeutically effective amount” will vary depending on the compound, the disease and its severity, the age, weight, physical condition and responsiveness of the mammal to be treated.

According to the present invention, in one embodiment, a therapeutically effective amount of vilazodone is an amount effective to treat depressive disorders. In another embodiment, a therapeutically effective amount is an amount effective to treat anxiety disorders. Other uses include, but are not limited to, the treatment of bipolar disorders, mania, dementia, substance-related disorders, sexual dysfunctions, eating disorders, obesity, fibromyalgia, sleeping disorders, psychiatric disorders, cerebral infract, tension, for the therapy of side-effects in the treatment of hypertension, cerebral disorders, chronic pain, acromegaly, hypogonadism, secondary amenorrhea, premenstrual syndrome, and undesired puerperal lactation. The effective amount of the drug for pharmacological action, and therefore the capsule or tablet strength, depends on the disease itself, e.g., in Major Depressive Disorder, the patient is initially given a 10 mg dose and the dosage is progressively increased to 20 mg once a day and to 40 mg once a day. Additional doses may be evaluated in clinical trials.

The term “pharmaceutically acceptable” means biologically or pharmacologically compatible for in vivo use in animals or humans, and preferably means approved by a regulatory agency of the Federal or a State government or listed in the U.S. Pharmacopeia or other generally recognized pharmacopeia for use in animals, and more particularly in humans.

The term “treat”, in all its verb forms, means to relieve or alleviate at least one symptom of a disorder in a subject, the disorder including for example, depressive disorders, anxiety disorders, bipolar disorders, mania, dementia, substance-related disorders, sexual dysfunctions, eating disorders, obesity, fibromyalgia, sleeping disorders, psychiatric disorders, cerebral infract, tension, for the therapy of side-effects in the treatment of hypertension, cerebral disorders, chronic pain, acromegaly, hypogonadism, secondary amenorrhea, premenstrual syndrome, and undesired puerperal lactation disease.

The term “treat” may mean to relieve or alleviate the intensity and/or duration of a manifestation of a disorder experienced by a subject in response to a given, without limiting, stimulus, or mood, loss of interest or pleasure, feelings of guilt or low self-worth, disturbed sleep or appetite, low energy and poor concentration. For example, in relation to depressive disorders, the term “treat” may mean to relieve or alleviate or mood, loss of interest or pleasure, feelings of guilt or low self-worth, disturbed sleep or appetite, low energy and poor concentration.

Within the meaning of the present invention, the term “treat” also denotes to arrest, delay the onset (i.e., the period prior to clinical manifestation of a disease) and/or reduce the risk of developing or worsening a disease.

The term “protect” means prevent delay or treat, or all, as appropriate, development or continuance or aggravation of a disease in a subject. Within the meaning of the present invention, the depressive disorders are associated with anxiety disorder. The term “treatment” means the act of “treating” as defined above.

The term “dose proportional” refers to the relationship between the dose of a drug and its bioavailability. For example, dose proportionality exists if twice as much of the same composition will deliver twice the drug and provide the same bioavailability (e.g., AUC) as one dose of the dosage form. The dose proportionality of the present invention applies to a wide range of doses as discussed in detail herein.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value. Where particular values are described in the application and claims, unless otherwise stated the term “about” means within an acceptable error range for the particular value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours.

The term “entry into a use environment” means contact of a formulation of the invention with the gastric or enteric fluids of the patient to whom it is administered, or with a fluid intended to simulate gastric fluid. As used herein, “use environment” refers to the stomach or other portion of the gastrointestinal tract intended as the site of major absorption locus for the drug.

The term “substantially the same dissolution stability” means similarity factor F₂ of greater than 50 as compared to a reference dissolution profile.

The term “dissolution stability” refers to the similarity of dissolution profiles (similarity factor greater than 50, in comparison to initial) obtained at different periods of storage at varying temperature and humidity conditions.

As used herein, the term “RSD” refers to Relative Standard Deviation and is obtained by dividing standard deviation of the blend by the absolute value of the mean. Relative Standard Deviation is expressed as percentage and referred to as “RSD %”. RSD % of less than 10 indicates excellent blend uniformity.

The term “salt” or “salts” may refer to any acid addition salts, including addition salts of free acids or addition salts of free bases. All of these salts (or other similar salts) may be prepared by conventional means. All such salts are acceptable provided that they are non-toxic and do not substantially interfere with the desired pharmacological activity.

The term “immediate release dosage form” as used herein refers to a composition that releases vilazodone from the composition in less than 6 hours following the oral dosing dependent or independent from the pH value.

The term “Cₘₐₓ” is defined as the observed maximum plasma concentration.

The term “Tₘₐₓ” is defined as the time of observed maximum plasma concentration.

The term “AUC” is defined as the area under the plasma concentration-time curve from time zero to time infinity.

The term “AUCT” is defined as the area under the plasma concentration-time curve from time zero to the last quantifiable concentration time point, calculated by linear trapezoidal rule. The term “t_{1/2}” is defined as the terminal disposition half-life.

The term “BMI” indicates body mass index.

The term “PK” indicates pharmacokinetic analysis.

### DETAILED DESCRIPTION OF THE INVENTION

In accordance with an aspect of the present invention, oral dosage forms are provided for administration of vilazodone, or one of its pharmaceutically acceptable salts, preferably its HCl salt, to a human, where the composition includes vilazodone in solid oral dosage forms. In particular, the pharmaceutical compositions of the present invention are directed to immediate release compositions of vilazodone, or one of its pharmaceutically acceptable salts.

Vilazodone (1-[4-(5-cyanoindol-3-yl)butyl]-4-(2-carbamoyl-benzofuran-5-yl)piperazine, and its physiologically acceptable salts thereof (disclosed, e.g., U.S. Pat. No. 5,532,241, incorporated herein by reference), belonging to the family of indole alkylamines, is a dual-acting serotonergic agent that combines antidepressant effects of selective serotonin-reuptake inhibitors (SSRI) with partial serotonin (5-HT)(1A)-receptor agonist activity. Vilazodone binds with high affinity to the serotonin reuptake site but not to the norepinephrine or dopamine reuptake site. As a result, vilazodone potently and selectively inhibits the reuptake of serotonin. Vilazodone, and its pharmaceutically acceptable salts, thereof (e.g., the HCl salt) is approved in the U.S. for the treatment of Major Depressive Disorder (MDD).

Vilazodone, polymorphic font's of vilazodone and methods for synthesis of vilazodone are disclosed in U.S. Pat. Nos. 5,532,241, 5,723,614, 5,977,112, 6,531,503, 6,509,475, 6,762,300, 6,900,212, 7,371,756, 7,381,726, 7,479,492, 7,642,261, 7,799,916, 7,834,020, 7,981,894, 8,193,195, 8,236,804, 8,318,744 and in U.S. Patent Application Publication 2013/01022616, disclosures of all of the above U.S. patents and patent application are hereby incorporated by reference in their entirety.

The present invention relates to an immediate-release, oral dosage formulation of vilazodone, comprising vilazodone and/or its pharmaceutically acceptable salts, preferably HCl salts, in combination with other excipients. The formulations may be in form of a tablet, granulation or capsule, where a tablet form is preferred. The formulations include a combination of excipients and are manufactured using a method that provides content uniformity, desirable tensile strength, and suitable disintegration and dissolution times. In some embodiments, the tablet include harness of about 5-14 kp, thickness of about 0.095-0.195″ friability not more than about 1.0% and disintegration time not more than about 2 minutes. Surprisingly, the formulation of the present invention provides these desirable properties despite the known problem of slow disintegration of tablets with increase in tablet hardness.

The present invention provides oral dosage forms that include vilazodone or a salt thereof, wherein the dosage form comprises 10 mg to 40 mg of vilazodone or a salt thereof and provides an in vivo plasma profile with a mean Tₘₐₓ of about 4 hours or more hours, t_{/2} of about 24 hours or more, a mean Cₘₐₓ of less than about 180 ng/ml and a mean AUC₀₋₂₄ of less than about 1800 ng h/ml.

In one embodiment of the present invention, vilazodone is used in form of pharmaceutically acceptable salts, thereof. Suitable salts of the compound include, but are not limited to, acid addition salts, such as those made with hydrochloric, methylsulfonic, hydrobromic, hydroiodic, perchloric, sulphuric, malonic, succinic, maleic, fumaric, tartaric, citric, benzoic, carbonic cinnamic, mandelic, methanesulfonic, methanesulfonic, hydroxyethanesulfonic, benezenesulfonic, p-toulene sulfonic, cyclohexanesulfamic, salicyclice, p-aminosalicyclic, 2-phenoxybenzoic, and 2-acetoxybenzoic acid. In a preferred embodiment, the salt is vilazodone hydrochloride (C₂₆H₂₇N₅O₂.HCl, MW 477.99).

It is possible to use any salts and free base forms of vilazodone, including polymorphs, hydrates, and solvates as well as amorphous forms of vilazodone. As used in the present specification, and claims, “vilazodone” is deemed to encompass both the free base and pharmaceutically acceptable salts, thereof. In preferred embodiments of the invention, the active ingredient is vilazodone hydrochloride.

The present invention provides immediate release pharmaceutical formulations that comprise vilazodone, and its pharmaceutically acceptable salts, thereof, and at least one pharmaceutically acceptable excipient, including one or more diluents, one or more fillers, one or more glidants, one or more lubricants, and optionally a coating.

The compositions of the present invention include about 0.01% and about 50% by weight of vilazodone. In one embodiment of the present invention, the compositions of the present invention includes about 0.05% and about 35% by weight of vilazodone. In another embodiment, the compositions of the present inventions include about 0.05% and about 10% by weight of vilazodone. The compositions as described herein may comprise at least one pharmaceutically acceptable excipient selected from a group comprising diluent, disintegration aid, glidant, lubricant, coloring agent and opacifying agent.

A diluent as described herein may be selected from a group comprising lactose, sucrose, glucose, dextrose, microcrystalline cellulose, dibasic calcium phosphate, calcium sulphate, mannitol, erythritol, lactilol, maltitol, xylitol, sorbitol, starch, and mixtures thereof. In some embodiments, the diluents may be present in an amount ranging from about 20% and about 80%, more preferably from about 40% and about 70% by weight of the composition. The preferred diluent is lactose monohydrate.

In some embodiments, the immediate release formulation as disclosed herein may exhibit a diluent content in a range selected from about 20% and about 70% by weight, about 20% and about 75% by weight, about 30% and about 70% by weight, about 30% and about 75% by weight, about 35% and about 80% by weight, about 35% and about 75% by weight, about 35% and about 70% by weight, about 30% and about 60% by weight, and about 30% and about 65% by weight.

A glidant as described herein may be selected from a group comprising water soluble excipient, hydrophilic polymers, silicon dioxide, colloidal silicon dioxide, microcrystalline cellulose, and combinations thereof. In particular, the glidant may be a water-soluble excipient. In some embodiments, the glidant may be present in an amount ranging from 0.05% and about 5%, more preferably from about 0.050% and about 1% by weight of the composition. The preferred glidant is colloidal silicon dioxide.

In some embodiments, the immediate release formulation as disclosed herein may exhibit a glidant content in a range selected from about 0.05% and about 4.5% by weight, about 0.05% and about 4% by weight, about 0.05% and about 3% by weight, about 0.1% and about 5% by weight, about 0.1% and about 4% by weight, about 0.1% and about 3% by weight, about 0.2% and about 5% by weight, about 0.2% and about 4% by weight, and about 0.2% and about 3% by weight.

A disintegration aid, or disintegrant, may be selected from a group comprising ion exchange resins, hydroxypropylcellulose, crospovidone, croscarmellose sodium, starches, pectins, alginates, surfactants, microcrystalline cellulose, sodium starch glycolate, and combinations thereof. In some embodiments, the disintegration aid may be present in an amount ranging from about 5% and about 30%, more preferably from about 5% and about 25% by weight of the composition. The preferred disintegration aid is microcrystalline cellulose.

In some embodiments, the immediate release formulation as disclosed herein may exhibit a disintegration aid content in a range selected from about 5% and about 25% by weight, about 5% and about 20% by weight, about 10% and about 20% by weight, about 10% and about 20% by weight, about 10% and about 15% by weight, about 15% and about 30% by weight, about 15% and about 20% by weight, and about 15% and about 10% by weight.

In particular embodiments, the immediate release formation may include one or more lubricants selected from a group comprising magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, zinc stearate, and combinations thereof. In some embodiments, lubricants may be added to the immediate release formulation in an amount resulting in lubricant content of between about 0.3% and about 10% by weight.

In some embodiments, the immediate release formulation as disclosed herein may exhibit a lubricant content in a range selected from about 0.1% and about 10% by weight, about 0.1% and about 5% by weight, and about 0.1% and about 2.5% by weight. In one such embodiment, lubricants may be present in the immediate release formulation, and the lubricant content may be selected from about 0.1%, 0.25%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% and 10% by weight. The optional coating is selected from Opadry® solution solids content.

Vilazodone tablet dosage forms set forth above, for example, may be prepared using the specific formulations and methods described further below.

TABLE 1
Vilazodone Tablet Formulation (40 mg Tablet)

| Ingredient | Amount (mg/Tablet) |
|---|---|
| Vilazodone HCl | 40.0 |
| Lactose Monohydrate | 277.2 |
| Microcrystalline Cellulose | 80.0 |
| Colloidal Silicon dioxide | 0.8 |
| Magnesium Stearate | 2.0 |
| Coating | 12.0 |
| Purified water | — |

During manufacturing of vilazodone HCl tablets, a higher than desirable level of tablet breakage was observed. According to the FDA approved dose regimen, vilazodone must be taken with food. If vilazodone is taken without food, inadequate drug concentrations may diminish the drug's effectiveness. Breakage of tablets comprising vilazodone HCl would also result in inadequate drug concentrations and, thus may diminish the drug's effectiveness. Manufacturing vilazodone HCl tablets using the method described herein resulted in vilazodone HCl tablets with no tablet breakage during the manufacturing process, including compression, coating or transportation of the tablets.

Accordingly, the present invention provides tablets comprising vilazodone or salts thereof with improved tablet breakage parameters. The method of preparing the immediate release formulation of vilazodone or salts with improved tablet breakage parameters is described below.

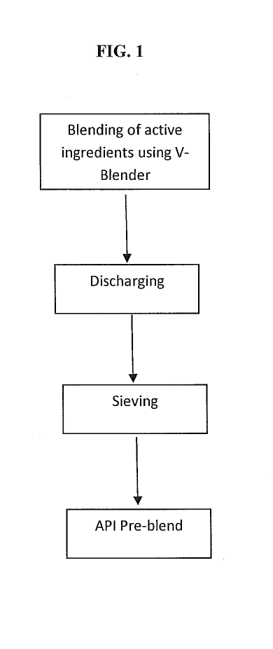

FIG. 1 of the drawings shows the process of pre-blending the active ingredients. Individual tablet ingredients are identified and weighed according to the strength of the tablet to be prepared. Lactose monohydrate, microcrystalline cellulose and vilazodone HCl are added to a conventional 3 cubic foot V-Blender equipped with a pin intensifier bar for pre-blending. Mixing time is adjusted based on revolutions per minute (rpm) of the blender. In some embodiments, the contents are mixed at about 20-50 rpm, more preferably at about 25 rpm for about 100-180 revolutions, most preferably at about 140 revolutions. The i-bar of the V-blender is set to an on state. After pre-blending, mixture is discharged and sieved manually or using Russell Finex sieve of 0.85 mm in US#20 mesh screen. Contents of the mixture are then discharged into clean, dry polyethylene containers or suitable stainless steel containers.

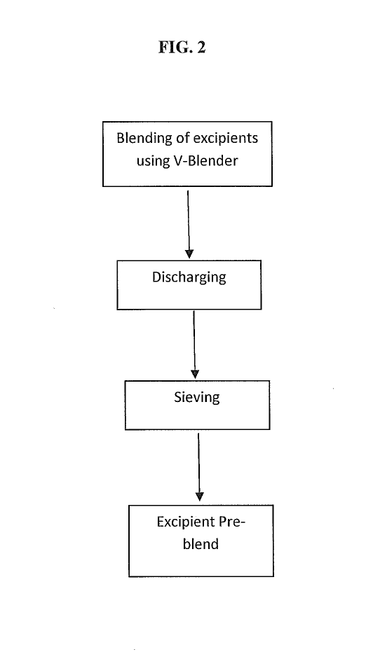

FIG. 2 of the drawings shows the process of pre-blending the excipients. Lactose monohydrate and microcrystalline cellulose are mixed in a conventional 3 cubic foot V-Blender with a pin intensifier bar. In some embodiments, the contents are mixed at about 20-50 rpm, more preferably at about 25 rpm for about 80-150 revolutions, most preferably at about 98 revolutions. The i-bar of the V-Blender is set to an off state. During excipient pre-blending aerosol is not added. After excipient pre-blending, the mixture is discharged and sieved manually or using Russell Finex sieve of 0.85 mm in US#20 mesh screen. Contents of the mixture are then discharged into clean, dry polyethylene containers or suitable stainless steel containers.

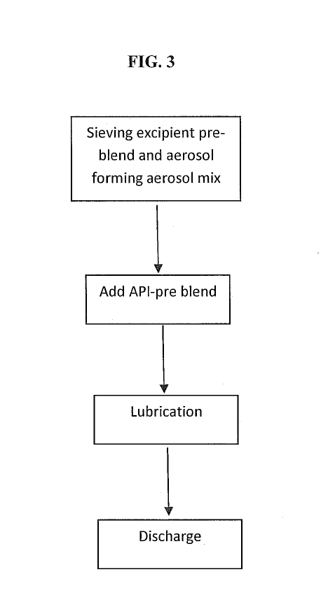

FIG. 3 of the drawings shows the process of final blending. Contents of the excipient pre-blend and aerosol are sieved using US#25 mesh screen. To this mixture, the pre-blended active ingredients and pre-blended excipients as shown in FIG. 1 and FIG. 2, respectively are added and blended in a 50 cubic foot V-Blender at around 10-25 rpm, preferably using a conventional 10 cubic foot V-Blender, preferably at about 19 rpm for about 150-300 revolutions, preferably at about 260 revolutions for about 5-35 minutes. The i-bar of the V-blender is set to an off state. The mixture is sampled at regular intervals using sufficient quantity of the blended mixture.

Magnesium stearate lubricant is added to the above blended mixture for final blending. Mixture is then blended using a conventional 50 cubic foot V-Blender, preferably using a conventional 10 cubic foot V-Blender, equipped with a pin intensifier bar for about 80-150 revolutions, preferably at about 130 revolutions. The i-bar of the V-Blender is set to an off state. After final blending, the contents are discharged from the V-Blender into clean, dry polyethylene containers or suitable stainless steel containers.

Tablet blend is then compressed using instrumented press Korsch XL 200 to prepare 10 mg, 20 mg and 40 mg vilazodone HCl tablets. The main compression force is set to desired hardness. In some embodiments, the desired hardness of 40 mg vilazodone HCl tablets is about 10-14 kp, preferably at about 12 kp. In other embodiments, the desired hardness of 20 mg vilazodone HCl tablets is about 8-12 kp, preferably at about 10 kp. In certain other embodiments, the desired hardness of 10 mg vilazodone HCl tablets is about 5-9 kp, preferably about 7 kp. Press speed is set to accommodate a similar dwell time as that of the press speed of the commercially available tablets, which exhibit high tablet breakage.

TABLE 2 below shows the compression in process parameters related to hardness of the commercial available vilazodone HCl tablets that exhibit high tablet breakage as well as the hardness of the vilazodone HCl tablets manufactured using instrumented press Korsch XL-200, as described herein, that resulted in no breakage.

TABLE 2

Average Hardness, Target (Range)

| Strength | High Tablet Breakage | No Tablet Breakage |
|---|---|---|
| 10 mg | 6 kp | 7 kp (5-9 kp) |
| 20 mg | 8 kp | 10 kp (8-12 kp) |
| 40 mg | 9 kp | 12 kp (10-14 kp) |

TABLE 3 shows the compression in process parameters achieved by the method of manufacturing described herein and as shown in FIGS. 1-3 that resulted in no breakage of vilazodone HCl tablets.

The weight range of vilazodone HCl tablets of present invention is set to about +/−3% to about +/−8% for individual tablets and about +/−1% to about +/−4% for average tablet weight. The tablet weight of commercially available tablets is about +/−10% for individual tablets and about +/−5% for average tablet weight. Number of rotations for friability testing is changed from 100 rotations used for commercially available vilazodone HCl tablets to about 300-400 rotations for vilazodone HCl of the present invention to subject the tablets to more stressed conditions.

TABLE 3
Compression in-process parameters of present invention

| | | | Average | |
|---|---|---|---|---|
| | Individual | Average | Hard- | |
| | Tablet weight | Tablet weight | ness | Thickness |
| | Target | Target | Target | Target |
| Strength | (Range) | (Range) | (Range) | (Range) |
| 10 mg | 100 mg | 10 mg | 7 kp | 0.110″ |
| | (95-105 mg) | (93-103 mg) | (5-9 kp) | (0.095-0.125″) |
| 20 mg | 200 mg | 200 mg | 10 kp | 0.140″ |
| | (190-210 mg) | (194-206 mg) | (8-12 kp) | (0.125-0.155″) |
| 40 mg | 400 mg | 400 mg | 12 kp | 0.180″ |
| | (380-420 mg) | (388-412 mg) | (10-14 kp) | (0.165-0.195″) |

| | |
|---|---|
| Friability | No more than 1% |
| Disinte- | No more than 2 minutes |
| gration | |
| Time | |

Compressed vilazodone HCl tablets are then coated using Opadry® solution solids content of about 10%-15% to an approximate weight gain of about 3%. The exhaust temperature is about 44-50° C. and the inlet temperature is adjusted to maintain 61-70° C. Air flow is maintained to about 600-800 CFM, and inlet humidity is maintained at about 5-20° C.

When weight gain of the tablets is achieved, spray and inlet air heat is turned off. Vilazodone HCl tablets are then coated with Opadry® solution solids content of about 10-15%. The finished tablets provide excellent content uniformity.

Vilazodone HCl tablets of the present invention may be subjected to in vitro dissolution studies according to U.S. Pharmacopeia or other generally recognized pharmacopeia for use in animals, and more particularly humans, or as referenced by FDA guidelines (“Dissolution Testing of Immediate Release Solid Oral Dosage Forms”, issued August 1997) to determine compliance with dissolution requirements.

In the dissolution testing guideline for immediate release profiles, material dissolves over a period of time, and its dissolution is measured at given intervals during this period. A minimum of three time points is recommended and generally cover early, middle and late stages of the dissolution profile. The last measurement should be no earlier than the time point where at least 80% of the drug is dissolved (FDA guidelines, “Dissolution Testing of Immediate Release Solid Oral Dosage Forms”, issued August 1997). Adequate sampling is important: for example, at 15, 30 and 45 minutes and every 15 minutes thereafter until 80% of the drug is released (FDA guidelines, “Dissolution Testing of Immediate Release Solid Oral Dosage Forms”, issued August 1997, at page 5). The preferred dissolution apparatus is USP apparatus I (basket) or II (paddle), used at recognized rotation speeds, e.g., 100 rpm for the basket and 50-75 rpm for the paddle (FDA guidelines, “Dissolution Testing of Immediate Release Solid Oral Dosage Forms”, issued August 1997, at page 5). In presence of minor changes to the drug dosage forms, a single point dissolution test may be adequate to ensure product dosing compliance. For major changes, a dissolution profile comparison performed under identical conditions for the product before and after the changes(s) is recommended. (FDA guidelines, “Dissolution Testing of Immediate Release Solid Oral Dosage Forms”, issued August 1997, at page 8).

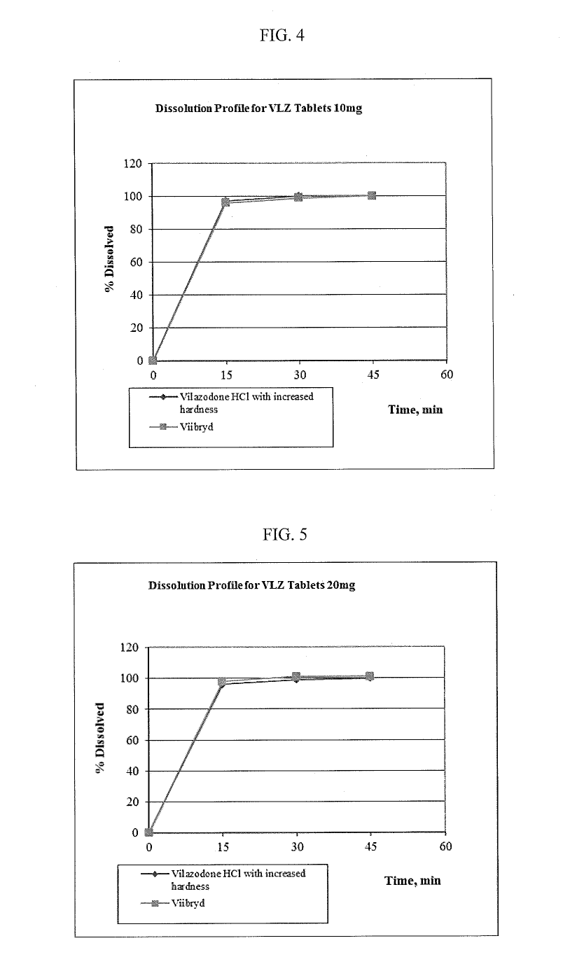

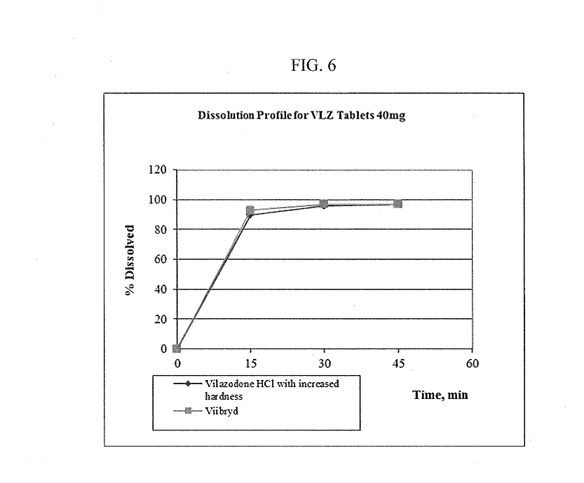

In vitro dissolution of commercially available dosage forms 40 mg, 20 mg and 10 mg vilazodone HCl is compared with the in vitro dissolution of the dosage forms 40 mg, 20 mg and 10 mg vilazodone HCl of present invention. Comparison of in vitro dissolution of vilazodone HCl tablets indicates substantially the same dissolution for both forms. A comparison of dissolution profiles of vilazodone HCl of the present invention and commercially available vilazodone HCl is provided in FIGS. 4-6.

Dissolution of immediate release vilazodone HCl dosage forms of the present invention and immediate release commercially available dosage forms of vilazodone HCl shows a similarity factor F₂ of greater than 50 (>50), which indicate excellent stability. High F₂ values (>50) obtained in comparison of dissolution rate of commercially available vilazodone HCl indicate excellent dissolution stability.

The term “similarity factor” or F₂ factor as used herein refers to one way of comparing dissolution profiles of two different products. (Multisource Pharmaceutical Products: Guidelines on Registration Requirements to establish Interchangeability, Quality Assurance and Safety: Medicines, Essential Drugs and Medicines Policy, World Health Organization, 1211 Geneva 27, Switzerland). This model independent mathematical approach compares the dissolution profile of the two products: test and reference (or two strengths, or pre- and post-approved products from the same manufacturer) and calculates percent (%) difference between two curves at each time point and measures the relative error between two curves. Tests are recommended to be performed under the same test conditions. The dissolution time points for both the profiles should be the same, for example for immediate release products e.g., 10, 15, 30, 45, 60 minutes. Only one time point should be considered after 85% dissolution of the reference product. The similarity factor F₂ should be computed using the equation:

F₂=50 log {[1+(1/n)Σₜ₌₁ⁿ(Rₜ−Tₜ)²]^{0.5}·100} Eq. 1

where Rₜ and Tₜ are the cumulative percentage of the drug dissolved at each of the selected In time points of the comparator (reference) and (test) product respectively. An F₂ value of 50 or greater (50-100) ensures sameness or equivalence of the two curves, and thus the performance of the two products.

The teen “dissolution stability” as used herein refers to the similarity of dissolution profiles (similarity factor greater than 50, in comparison to initial) obtained at different periods of storage at varying temperature and humidity conditions.

Although vilazodone HCl tablets of the present invention have increased hardness compared to commercial available vilazodone HCl tablets as described herein, it is surprisingly found that in vitro dissolution and dissolution stability of vilazodone HCl tablets of the present invention is substantially the same as that of the in vitro dissolution of commercially available vilazodone HCl (with lesser hardness). Despite the known problem of slow disintegration of tablets with an increase in tablet hardness, vilazodone HCl tablets of the present invention with increased hardness possess substantially the same dissolution stability as that of the commercially available, vilazodone tablets with lesser hardness.

In accordance with another aspect of the present invention vilazodone is extensively metabolized in the liver. Hepatic metabolism of vilazodone occurs through both non-cytochrome P450 (CYP) pathways (possibly carboxylesterase) and CYP3A4, with minor contributions from CYP2C19 and CYP2D6. M17, a butyric acid derivative of the N-dealkylation product of vilazodone, is a major metabolite of vilazodone and is considered pharmacologically inactive. Structure of M17, the metabolite of vilazodone is shown below.

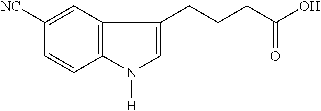 [SMILES: N#Cc1ccc2[nH]cc(CCCC(=O)O)c2c1]

Considering the extensive hepatic metabolism of vilazodone, it is critical to understand whether the pharmacokinetics are altered, and whether dose adjustments are necessary, in patients with various degrees of hepatic impairment.

Due to the extensive hepatic metabolism of vilazodone, the studies were performed in accordance with the present invention to determine whether its pharmacokinetic profile is altered, and dose adjustments necessary, in patients with mild, moderate, or severe hepatic impairment following administration of vilazodone HCl tablets with increased hardness. In these studies, all plasma pharmacokinetic parameter values in participants with mild and moderate hepatic impairment were similar to those in healthy controls, with mean values for Cₘₐₓ and AUC being within approximately 90% of the values observed in healthy matched participants.

In participants with severe hepatic impairment, mean Cₘₐₓ value of vilazodone was lower by approximately 25% compared with healthy participants, but values for AUC, Tₘₐₓ and t_{y}, were similar between groups. Consequently, drug accumulation with repeated dosing is likely to be similar between patients with severe hepatic impairment and those with normal hepatic function. In addition, although the mean AUC_{0-∞} of the primary metabolite M17 was approximately 42% lower in participants with severe hepatic impairment than in normal controls, the mean values for Cₘₐₓ, Tₘₐₓ, and t_{1/2} were similar, suggesting that no additional accumulation of M17 is expected following repeated dosing of vilazodone in patients with severe hepatic impairment.

Overall, the data from these studies suggest that there are no clinically significant effects of mild, moderate, or severe hepatic impairment on vilazodone pharmacokinetics, and the differences observed between the populations are unlikely to require dosage adjustments. Despite the fact that hepatic metabolism plays a major role in the clearance of vilazodone, its pharmacokinetics are not notably affected by hepatic impairment. This may be due to the large number of multiple metabolic pathways that can potentially be utilized during the elimination of vilazodone.

In accordance with another aspect of the present invention an open-label, multiple-dose, single-sequence study to evaluate the effect of steady-state carbamazepine XR, 400 mg once daily, a CYP3A4 substrate and inducer, on the pharmacokinetics of steady-state vilazodone (40 mg once daily, with increased hardness). Vilazodone was co-administered with carbamazepine XR (extended release formulation). Carbamazepine is an antiepileptic drug approved for the treatment of psychomotor and grand mal seizures is metabolized by CYP3A4, and is a CYP3A4 inducer.

The co-administration of vilazodone and carbamazepine XR, a CYP3A4 inducer, decreased mean steady-state vilazodone exposure by about 45%. The 90% CIs for the ratio (vilazodone plus carbamazepine XR vs vilazodone alone) of geometric means for AUC and Cₘₐₓ were not within the range of 80% to 125%, indicating a statistically significant effect of carbamazepine on the pharmacokinetics of vilazodone. The effect on the vilazodone metabolite M17 was highly variable with carbamazepine XR co-administration, describing the inter-individual variability in the inhibition and induction of CYP enzymes. Study also evaluated the pharmacokinetic parameters for M17 without carbamazepine XR co-administration.

### EXAMPLES

Two batches (batch 1, batch 2) of vilazodone HCl blend were manufactured. Batch 1 was used to manufacture 10 mg and 40 mg tablets, and batch 2 was used to manufacture 20 mg and 40 mg tablets. Pre-blend of active ingredients in batch 1 used a one cubic foot V-Blender.

### Example 1

The following process parameters were used to evaluate blend uniformity of final blends of batch 1 and batch 2. Batches were blended for 260 revolutions equivalent to 20 minutes in a 50 cubic foot V-Blender. Blend uniformity samples were taken at 200 revolutions and at 260 revolutions during blending. Batches were lubricated for 130 revolutions, equivalent to 10 minutes of lubrication time in a 50 cubic foot V-Blender. Blend uniformity samples were taken at 117 revolutions and 130 revolutions of the lubrication. TABLE 4 below shows the blend uniformity data of batches 1 and 2.

TABLE 4
Blend uniformity

| | Blending Process | | | |
|---|---|---|---|---|
| | Blending | Blending | Lubrication | Lubrication |
| | 200 | 260 | 117 | 130 |
| | revolutions | revolutions | revolutions | revolutions |
| Batch 1 | | | | |
| Mean | 99.5 | 99.5 | 99.7 | 99.4 |
| Range | 95.8-102.7 | 95.0-102.5 | 98.0-100.6 | 96.9-102.0 |
| RSD % | 2.3 | 2.1 | 0.8 | 1.5 |
| Batch 2 | | | | |
| Mean | 102.2 | 102.1 | 101.7 | 102.1 |
| Range | 93.5-114.2 | 97.5-107.9 | 98.7-105.4 | 99.8-105.8 |
| RSD % | 5.2 | 3.3 | 2.3 | 2.0 |

RSD % of less than 10 indicates excellent blend unifolinity. Blend uniformity is good after about 260 revolutions of blending and after about 130 revolutions of lubrication using a 50 cubic foot V-Blender.

### Example 2

During a comparative compression study, no breakage of tablets of the present invention was observed. The following process parameters were used to evaluate compression and hardness parameters of vilazodone HCl tablets of the present invention. The press speed range was set to accommodate similar press time as that of commercially available vilazodone HCl tablets and as referenced by FDA guidelines (“Dissolution Testing of Immediate Release Solid Oral Dosage Forms”, issued August 1997). Compression challenges were performed for 10 mg, 20 mg and 40 mg vilazodone HCl dosage forms according to batch 1 and batch 2 of the present invention. Results of the compression challenge for batch 1 and batch 2 are shown below in TABLES 5-7.

TABLE 5
Compression Challenge Results including Dissolution Data
for Vilazodone HCl 10 mg Tablets of Present Invention.

| | Press Speed | | | |
|---|---|---|---|---|
| | Low/Low/ | Low/High/ | High/Low/ | High/High/ |
| | 5-6 kp | 8-9 kp | 5-6 kp | 8-9 kp |
| Average | 5.7 kp | 8.5 kp | 5.5 kp | 8.2 kp |
| Tablet | | | | |
| Hardness | | | | |
| Disintegration | 1 minute | 1 minute | 45 seconds | 1 minute |
| Time (37° C.) | 8 seconds | 5 seconds | | 5 seconds |
| Friability(400 | 0.5% | 0.3% | 0.4% | 0.3% |
| rotations) | | | | |
| Compression | 5-6 | 6-10.5 | 5-6 | 6-10.5 |
| Force (kN) | | | | |

TABLE 6
Compression Challenge Results including Dissolution Data
for Vilazodone HCl 20 mg Tablets of Present Invention.

| | Press Speed | | | |
|---|---|---|---|---|
| | Low/Low/ | Low/High/ | High/Low/ | High/High/ |
| | 8-9 kp | 11-12 kp | 8-9 kp | 11-12 kp |
| Average | 8.4 kp | 11.3 kp | 8.9 kp | 11.1 kp |
| Tablet | | | | |
| Hardness | | | | |
| Disintegration | 54 seconds | 1 minute | 1 minute | 1 minute |
| Time (37° C.) | | 50 seconds | | 32 seconds |
| Friability(400 | 0.4% | 0.2% | 0.2% | 0.3% |
| rotations) | | | | |
| Compression | 8-10 | 10-13.4 | 8-10 | 10-13.4 |
| Force (kN) | | | | |

TABLE 7
Compression Challenge Results including Dissolution Data
for Vilazodone HCl 40 mg Tablets of Present Invention.

| | Press Speed | | | |
|---|---|---|---|---|
| | Low/Low/ | Low/High/ | High/Low/ | High/High/ |
| | 10-11 kp | 13-14 kp | 10-11 kp | 13-14 kp |
| Average | 10 kp | 13.3 kp | 10.5 kp | 14.3 kp |
| Tablet | | | | |
| Hardness | | | | |
| Disintegration | 42 seconds | 1 minute | 54 seconds | 1 minute |
| Time (37° C.) | | 18 seconds | | 26 seconds |
| Friability(400 | 0.6% | 0.4% | 0.5% | 0.4% |
| rotations) | | | | |
| Compression | 9-14 | 14-17 | 9-14 | 14-17 |
| Force (kN) | | | | |

### Example 3

After compression, vilazodone HCl tablets were subjected to material handling study. Core tablets were packed into one stainless steel round drum of about 40 gallons in volume and conditions were stimulated to replicate normal handling conditions. After material handling, the core tablets were visually inspected to determine if there was any tablet breakage. No tablet breakages or defects were observed.

### Example 4

Dissolution stability is provided in FIGS. 4-6. High F₂ values (>50) obtained in comparison of dissolution rate of commercially available vilazodone HCl indicate excellent dissolution stability.

A batch of 40 mg, 20 mg, and 10 mg vilazodone HCl dosage forms of the present invention were tested using a suitable dissolution apparatus with rotating paddle such as “apparatus 2” with a rotating paddle as defined by USP. The paddle speed was set to 60 rpm and the dissolution medium was 0.1% acetic acid. The pH values for dissolution testing for 40 mg, 20 mg, and 10 mg vilazodone HCl dosage forms are pH 4.5, pH 1.2, pH 6.8, respectively. Results of % dissolution of 10 mg, 20 mg and 40 mg dosage form's are listed below in TABLE 8.

TABLE 8

| | | |
|---|---|---|
| | Vilazodone HCl with | Vilazodone |
| Dissolution time | increased hardness | commercially available |
| (minutes) | (% dissolved) | (% dissolved) |

Vilazodone HCl 10 mg (coated)

| | | |
|---|---|---|
| 0 | 0 | 0 |
| 15 | 97 | 96 |
| 30 | 100 | 99 |
| 45 | 100 | 100 |

Vilazodone HCl 20 mg (coated)

| | | |
|---|---|---|
| 0 | 0 | 0 |
| 15 | 96 | 98 |
| 30 | 99 | 101 |
| 45 | 100 | 101 |

Vilazodone HCl 40 mg (coated)

| | | |
|---|---|---|
| 0 | 0 | 0 |
| 15 | 90 | 93 |
| 30 | 96 | 97 |
| 45 | 97 | 97 |

Although vilazodone HCl tablets of the present invention have increased hardness compared to commercial available vilazodone HCl tablets, it is surprisingly found that in vitro dissolution of vilazodone HCl tablets of the present invention is substantially the same as that of the in vitro dissolution of commercially available vilazodone HCl. Despite the known problem of slow disintegration of tablets with increase in tablet hardness, vilazodone HCl tablets of the present invention with increased hardness possess substantially the same dissolution stability as that of the commercially available, vilazodone tablets with lesser hardness.

Dissolution of immediate release vilazodone HCl dosage forms of the present invention and immediate release commercially available dosage font's of vilazodone HCl shows a similarity factor F₂ of greater than 50 (>50), which indicate excellent stability. Dissolution stability is provided in FIGS. 4-6. FIGS. 4-6 show 80% of vilazodone HCl being released at first 30 minutes after administration of the drug product.

FIGS. 4-6 show high F₂ values (>50) obtained when dissolution rate of vilazodone HCl of the present invention is compared with the dissolution rate of commercially available vilazodone HCl, which indicate excellent dissolution stability. Additionally, F₂ value of 50 or greater (50-100) ensures sameness or equivalence of the two curves, and thus the performance of the two products.

### Example 5

### Pharmacokinetic Parameters of the Vilazodone HCl Tablets

This example compares bioavailability of vilazodone HCl tablet formulation at 20 mg level used in phase III clinical studies. Vilazodone HCl tablets are administered to individuals/study groups. Current clinical use of vilazodone HCl as a marketed product and in clinical trials utilize a dosing regimen with a starting dose of 10 mg once daily for seven days, followed by 20 mg once daily for additional seven days and then increased to 40 mg once daily.

The following pharmacokinetic parameters were assessed following a single dose administration of vilazodone HCl tablet dosage form to a subject: Cₘₐₓ, Tₘₐₓ, AUC, AUCT, and t_{1/2}. Estimates of the terminal half-life (t₁₇₂) were calculated using the following equation:

T_{1/2}=0.693/λ_{z} Eq. 2

where λ_{z} is the terminal elimination rate constant.
The area under the plasma concentration-time curve from time zero to infinity was calculated according to the following equation:

AUC_{0-∞}=AUC₀₋ₜ+Clast/λ_{z} Eq. 3

where Cₗₐₛₜ is the last measurable concentration.

### Subjects and Methods

An open, randomized, single dose, two session cross over study in healthy volunteers was performed in healthy male and female subjects aged 18-55 years. All subjects had a body weight >50 kg and BMI within the range 19-29.9 kg/m² inclusive. 30 patients were enrolled in the study to ensure evaluable data from at least 21 subjects. Subjects were screened at the start of the study for medical history evaluation, physical examination, clinical laboratory evaluations, including urinalysis, female pregnancy test, urine drug screen and clinical safety tests. Exclusion criteria for the study included a definite or suspected personal or family history of adverse reactions or hypersensitivity to the trial drug or to drugs with a similar chemical structure, a history of psychiatric illness, a known or suspected history of alcohol consumption defined as an average daily intake of greater than three units or a weekly intake of more than 21 units for males and a daily intake of greater than two units or a weekly intake of more than 14 units for females (one unit was equivalent to 250 mL beer, a glass of wine or a measure of spirits), subject had received prescribed medication within 14 days prior to the first dosing day, subject had received non-prescription drug (especially medicines containing paracetamol or cimetidine) or herbal medicines (especially St Johns Wort) within 48 hours prior to the first study day, a history or presence of gastrointestinal, hepatic or renal disease or any other condition known to interfere with the absorption, distribution, metabolism or excretion of drugs, subject had consumed grapefruit or grapefruit juice within seven days of the first study day, subject had been exposed to more than three new chemical entities within 12 months prior to the first dosing session, subject had participated in a trial with a different new chemical entity within 112 days prior to the start of the study, subject had participated in a trial with any drug within 84 days before the start of the study, if participation in the study would have resulted in the subject having donated more than 1500 mL of blood (males) or 1000 mL of blood (females) in the previous 12 months.

The subjects received the following treatments in a randomized order. Doses were administered with food in the clinic all dosing days. Treatment regimens included the following: Treatment A: 20 mg phase II capsule; Treatment B: 20 mg phase III tablet. A total of 30 subjects were screened and entered in the study and of the 30 subjects, three subjects withdrew from the study after receiving study medications. Blood samples (approximately 3 mL) were collected into tubes containing EDTA prior to dosing and nominally at 1, 2, 3, 4, 5, 6, 8, 10, 14, 24, 32, 48, 72, 96, 120, 144 and 168 hours following dosing at each study session. The blood samples were placed on crushed water ice and then centrifuged at 3000 rpm for 10 minutes at +4° C. The resultant plasma was placed in polypropylene tubes and frozen immediately and stored at approximately −20° C. or colder until shipment. All plasma samples were stored frozen at approximately −20° C. prior to analysis.

Plasma samples were analyzed by a method based on solid phase extraction (SPE), followed by HPLC/MS/MS analysis employing positive-ion electrospray ionization. The lower limit of quantification was 0.5 ng/mL based on a 100 μL aliquot of plasma. Quality control (QC) samples were assayed with each batch of samples against separately prepared calibration standards. The results of the QC samples were used to assess the day-to-day performance of the assay.

The pharmacokinetic parameters calculated for each subject were: the maximum observed plasma concentration (Cₘₐₓ) and the time to reach Cₘₐₓ (Tₘₐₓ). The area under the plasma concentration-time curve from time zero to the last quantifiable concentration, AUC₍₀₋ₜ₎, was determined using the linear trapezoidal rule for each incremental trapezoid up to Cₘₐₓ, and the log trapezoidal rule for each trapezoid thereafter. The terminal phase rate constant (2) was determined using unweighted linear least squares regression analysis of those points visually assessed to be in terminal phase. The pharmacokinetic parameters are provided in Table 9.

Safety parameters (adverse events, vital signs, clinical laboratory evaluations and ECG parameters) were summarized for all subjects. A total of 23 subjects experienced treatment-emergent adverse events when taking 20 mg capsule and 22 subjects experienced TEAEs when taking 20 mg tablet. The same number of treatment-emergent adverse events (n=48), was observed in each regimen Adverse events were reported as either mild (80 AEs) or moderate (23 AEs) in severity. The incidence of AE severity across treatment group was constructed and there do not appear to be differences in AE severity between the treatment groups. There were no severe AEs, deaths or serious AEs during the study. At the end of the study, three adverse events were ongoing. One subject with mild back pain, another subject with moderate myalgia and yet another with mild pain. None of these AEs was related to study medication. All other reported AEs had resolved at the end of the study.

TABLE 9
Pharmacokinetic parameters of 20 mg Vilazodone HCl

| Parameter [units] | Phase II capsule (A) | Phase III tablet (B) |
|---|---|---|
| AUC(0-t) [ng · h/mL] | 592 (407-1134) | 629 (376-1286) |
| Cmax [ng/mL] | 29.0 (17.2-48.4) | 32.1 (20.7-58.1) |
| Tmax [hours] | 5.98 (4.00-8.08) | 5.01 (2.02-10.0) |
| AUC(0-∞) [ng · h/mL] | 602 (465-856) | 544 (402-632) |
| T_{1/2} [hours] | 24.8 (19.8-30.6) | 22.4 (13.2-40.0) |

Vilazodone HCl tablets exhibited a consistent PK profile across bioavailability, bio equivalence and PK studies with the median time of maximum plasma concentration (Tₘₐₓ) occurring at about 4 hours or more, preferably at 4 hours to 6 hours post administration, and decline with a terminal half-life at about 20 hours or more, preferably at about 25 hours post administration. The Cₘₐₓ values showed dose-proportional pharmacokinetics over the dose range of 10 mg to 40 mg when administered as single doses and with repeated by once a day dosing in healthy subjects. The mean AUC₀₋₂₄ hours at steady state produced by 40 mg once a daily dose was less than about 1800 ng·hr/ml, preferably at about 1645 ng·hr/ml.

Consistent with dose proportional pharmacokinetics, the oral clearance of vilazodone HCl tablets is independent of dose, having an average value of about 31 L/hr over a dose range of 20 mg to 60 mg, and a median value of 21 L/hr. Absolute bioavailability of vilazodone HCl tablets with food is 72%. Administration with food increases oral availability, where Cmax is increased by approximately 147%460% and AUC is increased by approximately 64-85%.

### Example 6

### Pharmacokinetics and Safety of Vilazodone in Subjects with Hepatic Impairment

Vilazodone is extensively metabolized in the liver. Hepatic metabolism of vilazodone occurs through both non-cytochrome P450 (CYP) pathways (possibly carboxylesterase) and CYP3A4, with minor contributions from CYP2C19 and CYP2D6 [2, 3]. M17, a butyric acid derivative of the N-dealkylation product of vilazodone, is a major metabolite of vilazodone and is considered pharmacologically inactive. The structure of M17 is shown below.

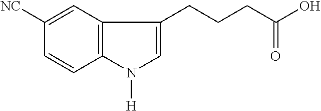 [SMILES: N#Cc1ccc2[nH]cc(CCCC(=O)O)c2c1]

Considering the extensive hepatic metabolism of vilazodone, it is critical to understand whether the pharmacokinetics are altered, and whether dose adjustments are necessary, in patients with various degrees of hepatic impairment.

The pharmacokinetics, tolerability, and safety of vilazodone were investigated in two separate trials involving participants with hepatic impairment, compared with healthy controls. An Open-label, parallel-group, single-dose pharmacokinetic studies, a single, oral dose of vilazodone (20 mg with increased hardness) was administered to participants with mild, moderate, or severe hepatic impairment or individually matched controls. Outcomes included pharmacokinetic parameters and safety (vital signs, clinical laboratory evaluations, ECG, and adverse events [AEs]). Plasma samples were analyzed using validated LC/MS/MS methods.

Data from two, open-label, parallel-group, single-dose pharmacokinetic studies investigated single-dose vilazodone (20 mg with increased hardness) in participants with hepatic impairment and normal hepatic function. The first study (Study 1) included participants with mild or moderate hepatic impairment and healthy controls. The second study (Study 2) included participants with severe hepatic impairment or healthy participants. These studies were both designed in accordance with the FDA guidance for evaluation of pharmacokinetics in the presence of hepatic impairment. The differences between the study protocols were minimal, so both methodologies are described together, with any differences noted below.

Participants were men and women, 18 to 70 years of age, with a body mass index (BMI) of 18-42 kg/m². Women were not pregnant or breastfeeding and agreed to utilize a medically accepted form of contraception during the study (as did men in Study 2). In Study 2, participants were also required to be non-smokers or light smokers (fewer than 10 cigarettes per day; there was no stipulation in Study 1) and to have a sitting pulse rate of 50-100 bpm at screening and prior to dosing on Day 1. Healthy control participants had normal hepatic function and were in clinically good health, according to physical examination, medical history, electrocardiogram (ECG), and laboratory results. Each healthy control was matched with a hepatically impaired participant. Control participants in the Study 1 were matched with a hepatically impaired individual according to age (±10 years), sex, and BMI (±15%). Control participants in Study 2 were matched with a hepatically impaired individual according to age (±5 years), sex, and weight (±10%).

Participants with hepatic dysfunction were classified according to the Child-Pugh hepatic dysfunction staging system as having mild (5-6 points) or moderate (7-9 points) hepatic impairment (Study 1), or severe impairment (10-15 points; Study 2). Participants with severe impairment also had chronic liver disease and/or compensated cirrhosis, as evidenced by liver biopsy, computerized tomography or ultrasonic evidence of hepatic fibrosis, clinical evidence of chronic liver disease, or a colloid shift on a liver-spleen scan. Exclusion criteria included the following: a clinical diagnosis or history of depression; treatment with antidepressants, anxiolytics, psychoactive medications (including herbal supplements), other serotonergic drugs, or drugs that inhibit CYP3A4 (eg, ketoconazole, diltiazem, macrolide antibiotics); a hypersensitivity to vilazodone or other SSRIs or 5-HT agonists; history or evidence of alcohol or substance abuse, cancer, human immunodeficiency virus, hepatitis B (Study 1 and healthy controls, both studies) hepatitis C (healthy controls, both studies), or other clinically significant illnesses or medical conditions. Participants with severe hepatic impairment were excluded if they had bleeding diathesis, advanced ascites requiring large-volume paracentesis within 1 week of dosing, esophageal bleeding within the previous 8 weeks, history of portosystemic shunt or hepatic transplant, or evidence of hepatic encephalopathy, hepatorenal syndrome, or other evidence of rapid hepatic deterioration.

Participants were not allowed to have previously taken vilazodone or participated in any clinical investigation requiring repeated blood draws within 60 days of dosing, or donated blood or plasma within 60 or 30 days, respectively. Aspirin, non-steroidal anti-inflammatory drugs, and other agents that affect coagulation were not allowed within 28 days of dosing; other over-the-counter and prescription drugs (other than those prescribed as therapy for hepatic conditions in patients with hepatic impairment) were prohibited within 14 days of dosing. Grapefruit-containing products, broccoli, and cauliflower were not allowed within 7 days, nor were alcohol within 72 hours or caffeine within 48 hours of dosing. All participants provided written informed consent before study procedures, and the study protocols were approved by an institutional review board at each investigational center.

Both studies used an open-label, single-dose trial design to investigate the pharmacokinetics of orally administered vilazodone 20 mg tablets with hardness of more than 8 kp and less than 12 kp, preferably about 10 kp as shown in Table 3 (no tablet breakage). The studies included a screening period (within 3-4 weeks prior to dosing), a 5-night inpatient treatment period (day −1 through day 5), 2 outpatient visits (days 6 and 7), and, in Study 2, a follow-up visit (day 14±1 day). The screening visit consisted of a medical history evaluation (including prior and concomitant medications), a complete physical examination (blood pressure, pulse rate, temperature, height, weight, and respiration rate), clinical laboratory evaluations (hematology, chemistry, urinalysis, urine microscopy [Study 1 only], serology, and a screen for drugs of abuse), a 12-lead ECG, an assessment of suicidality using the Columbia-Suicide Severity Rating Scale (C-SSRS; Severe Study), and a serum pregnancy test for women. These parameters were also assessed at various times throughout the two studies.

Vilazodone 20 mg was administered in the morning of day 1, following an overnight fast, as a single 20 mg tablet taken with 240 mL of water after a light standard breakfast. To assess the plasma concentrations of vilazodone and its primary inactive metabolite M17, blood samples (6 mL each) were collected by a qualified phlebotomist into prechilled 6-mL purple-topped Vacutainer tubes (containing K₂EDTA as an anticoagulant). Samples were collected at time 0 (predose) and at 0.5, 1, 2, 3, 4, 5, 6, 8, 10, 12, 16, 24, 36 (Study 2 only), 48, 72, 96, 120, and 144 hours postdose. In both studies, additional samples (6 mL each) were collected to determine vilazodone protein binding: in Study 1, these samples were collected in duplicate at predose hour 0 and postdose hours 6 and 12; in Study 2, the samples were collected at predose hour 0 and postdose hours 4 and 12, with an additional sample (13 mL) collected at hour 0 for the determination of albumin concentration. Blood samples were centrifuged within 30 minutes (2500×g for 10 minutes at 4° C.), flash-frozen, and shipped for analysis.

Urine samples from pooled urine collections for pharmacokinetic assessments in Study 1 were collected over the following post-dose periods: hours 0-3, 3-6, 6-9, 9-12, 12-24, 24-48, 48-72, and 72-96.

The plasma concentrations of vilazodone (Studies 1 and 2) and its metabolite M17 (Study 2), vilazodone concentrations in urine (Study 1), and protein binding were determined using liquid chromatography with tandem mass spectrometry (LC/MS/MS) methodologies that have been validated for the following ranges: plasma vilazodone, 0.75 to 250 ng/mL (Study 1) and 0.5 to 250 ng/mL (Study 2); plasma M17, 0.25-125 ng/mL; urine vilazodone, 1.0-200 ng/mL; vilazodone protein binding, high range (sample compartment) 2.7-108 ng/mL, low range (buffer compartment) 0.1-5.4 ng/mL. The extent of vilazodone binding to plasma proteins was determined using equilibrium dialysis.

### Pharmacokinetic Analyses

The principal parameters describing the pharmacokinetics of vilazodone (both studies) and its metabolite M17 (Study 2) were obtained using non-compartmental analysis with the software program WinNonlin (version 5.2.1) or Phoenix WinNonlin (version 6.1). These parameters included maximum plasma concentration (Cₘₐₓ); Tₘₐₓ; area under the plasma concentration-time curve from time 0 to the last measurable concentration (AUC₀₋ₜ), or time 0 to infinity (AUC_{0-∞}); oral clearance (CL/F), calculated as dose/AUC_{0-∞}; terminal elimination rate constant (λ_{z}); t₁₁₂; free fraction in plasma (fₚ); apparent free drug clearance (CLu/F), calculated as (CL/F)/fₚ; and volume of distribution (Vz/F), calculated as dose/(λz·AUC_{0-∞}). In Study 2, the ratio of M17/Vilazodone was also determined, calculated as the M17/vilazodone AUC_{0-∞} ratio. In Study 1, the area under the plasma concentration-time curve from time 0 to 24 hours (AUC₀₋₂₄), the amount of vilazodone recovered in urine (Au), percentage of dose recovered in urine, and renal clearance (CLr), estimated by Au/AUC₀₋₉₆, were also calculated.

### Safety Assessments

Safety assessments included vital signs, clinical laboratory evaluations, ECG, and the collection of adverse events (AEs). An AE was defined as any untoward medical event occurring in a participant administered study drug, irrespective of whether it had a causal relationship to the drug (e.g., any unfavorable and unintended sign, abnormal laboratory finding, symptom, or disorder temporally associated with study drug, whether or not considered related to study drug). AEs were recorded at all study visits and as they were reported spontaneously by the participants, up to 30 days after the vilazodone dose. Participants were asked about their well-being at each visit.

### Statistical Analysis

The sample sizes for both studies were based on clinical and practical considerations and not formal statistical power calculations. The Safety Population included all participants who received vilazodone and had at least 1 safety measure recorded. The PK Population included all participants who received vilazodone, completed the study, and had evaluable pharmacokinetic parameters; participants in Study 2 also were required to be matched with another healthy participant who completed pharmacokinetic evaluations.

In the Study 1, an analysis of variance (ANOVA) was performed on the natural logarithms of AUC₀₋ₜ, AUC_{0-∞}, and Cₘₐₓ, with study group (mild hepatic impairment, moderate hepatic impairment, and matched healthy control groups) as a fixed effect. Comparisons of each hepatic impairment group versus the matched healthy control group were made using appropriate contrast statements.

In the Study 2, pharmacokinetic parameters were compared by means of an ANOVA model using Phoenix WinNonlin (Version 6.1). A linear mixed effects model with study group as a fixed effect was used. Statistical inference was based on log-transformed values for the Cₘₐₓ, AUC₀₋ₜ, AUC_{0-∞}, and metabolite ratio (MR) parameters.

For both studies, the point estimates and the 2-sided 90% confidence intervals for the LS mean difference of log-transformed parameters between each hepatically impaired group verses the control group was exponentiated to obtain estimates for ratios of LS geometric means on the original scale.

Safety assessments were summarized without statistical comparisons. The last assessment made before the first dose of study drug was used as a baseline for all subsequent analyses.

### Results

In Study 1, 33 participants were enrolled and received study medication, and 32 were evaluated for the pharmacokinetic analysis. One participant in the healthy control group for mild impairment was determined to have mild hepatic impairment after dosing, and was withdrawn from the study; this participant was included in the Safety Population but not the PK Population. In Study 2, 16 participants were enrolled and received study medication, with all 16 completing the study and being evaluated for the pharmacokinetic analysis.

The baseline characteristics of participants with mild, moderate, and severe hepatic impairment and their matched healthy controls are shown in Table 10. Baseline serum albumin levels were lower in participants with severe hepatic impairment than in matched healthy controls (2.9±0.4 vs 4.2±0.3 g/dL; normal range: 3.2-5.5 g/dL), which is not unexpected in patients with hepatic impairment.

TABLE 10
Demographics and Baseline Characteristics (Safety Population) of Study 1 (Mild-
Moderate Hepatic Impairment) and Study 2 (Severe Hepatic Impairment)

| | Mild | | Moderate | | Severe | |
|---|---|---|---|---|---|---|
| | Impairment | Control | Impairment | Control | Impairment | Control |
| | n = 8 | n = 9 | n = 8 | n = 8 | n = 8 | n = 8 |
| Parameter | | | | | | |
| Age, years | 48.5 ± 6.2 | 44.3 ± 7.9 | 57.8 ± 2.2 | 55.3 ± 5.8 | 51.5 ± 5.9 | 51.4 ± 5.4 |
| Min-Max | 39.9-55.1 | 29.0-53.0 | 54.4-60.9 | 47.4-64.4 | 43-60 | 42-59 |
| Male, n (%) | 5 (62.5) | 6 (66.7) | 6 (75.0) | 6 (75.0) | 6 (75.0) | 6 (75.0) |
| Race | | | | | | |
| White, n (%) | 4 (50.0) | 8 (88.9) | 8 (100.0) | 6 (75.0) | 8 (100.0) | 6 (75.0) |
| Black, n (%) | 4 (50.0) | 1 (11.1) | 0 | 2 (25.0) | 0 | 2 (25.0) |
| Height, cm | 172.4 ± 9.6 | 175.6 ± 4.9 | 175.5 ± 8.9 | 171.4 ± 7.5 | 176.4 ± 11.1 | 179.3 ± 11.7 |
| Min-Max | 163-188 | 168-183 | 165-188 | 155-180 | 166-199 | 160-193 |
| Weight, kg | 80.6 ± 15.6 | 85.3 ± 15.1 | 83.5 ± 16.2 | 80.5 ± 13.8 | 99.2 ± 21.9 | 98.2 ± 17.8 |
| Min-Max | 58-112 | 62-109 | 66-110 | 65-111 | 68.0-137.7 | 69.5-126.3 |
| BMI, kg/m² | 27.1 ± 3.8 | 27.7 ± 4.8 | 27.0 ± 5.0 | 27.4 ± 4.4 | 31.5 ± 3.7 | 30.4 ± 3.1 |
| Min-Max | 22-32 | 21-36 | 23-38 | 24-38 | 24.7-35.9 | 26.1-35.1 |
| Serum | 3.9 ± 0.5 | 4.5 ± 0.3 | 3.4 ± 0.6 | 4.3 ± 0.2 | 2.9 ± 0.4 | 4.2 ± 0.3 |
| albumin, | | | | | | |
| g/dL | | | | | | |
| Min-Max | 3.0-4.5 | 3.9-4.9 | 2.5-4.3 | 4.1-4.7 | 2.2-3.4 | 3.8-4.6 |

### Pharmacokinetic Parameters

Data from all 32 matched participants in the Mild-Moderate Study were included in the final pharmacokinetic analysis. In Study 2, 4 participants with severe hepatic impairment vomited within 4 hours of dosing; pharmacokinetic parameters were determined for these participants, but their data were excluded from the statistical comparison analysis.

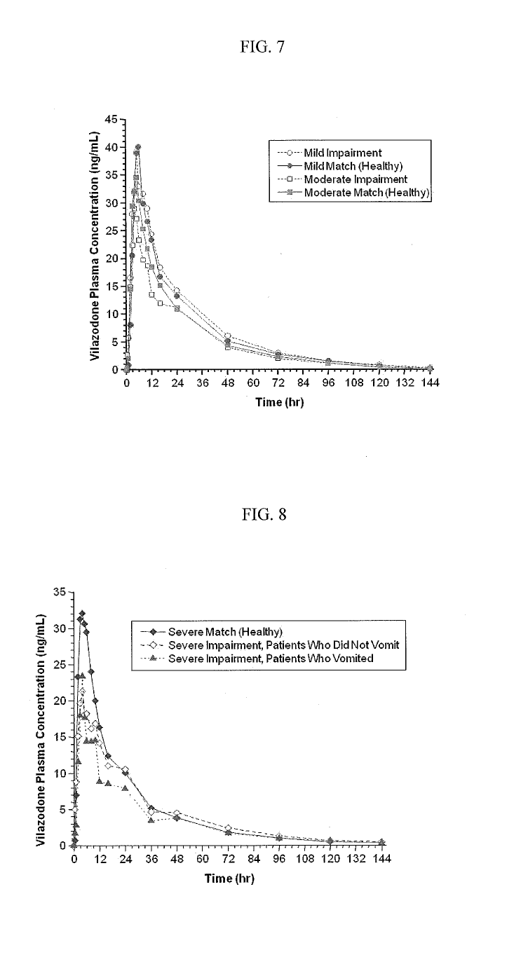

Mean plasma vilazodone concentration-time profiles were similar between participants with mild or moderate hepatic impairment and their matched healthy controls as shown in FIG. 7. In participants with severe hepatic impairment, mean plasma concentrations at time points surrounding Tₘₐₓ were lower than in healthy controls; plasma concentrations in the 4 participants who vomited were lower than those in the 4 participants who did not vomit as shown in FIG. 8.

In participants with severe hepatic impairment, the mean Cₘₐₓ value was lower than those in healthy controls by approximately 25%, AUC, Tₘₐₓ and t_{1/2} values were similar. In participants with mild and moderate hepatic impairment, all values were similar to those in healthy controls. The ratios (hepatically impaired/normal control) of the geometric mean values for Cₘₐₓ and AUC are shown by level of hepatic impairment in Table 11.

TABLE 11
Vilazodone Pharmacokinetic Parameter Comparisons between Hepatically
Impaired and Healthy Control Groups (PK Population)

| | Ratio of | |
|---|---|---|
| | Geometric | |
| Comparison | Means (%)* | 90% CI |
| Cₘₐₓ | | |
| Mild impairment/control | 97.8 | 72.4-132.3 |
| Moderate impairment/control | 97.2 | 71.9-131.4 |
| Severe impairment^{†}/control | 75.2 | 54.8-103.2 |
| AUC₀₋ₜ | | |
| Mild impairment/control | 97.7 | 71.3-133.8 |
| Moderate impairment/control | 89.5 | 65.3-122.6 |
| Severe impairment^{†}/control | 96.9 | 69.3-135.6 |
| AUC_{0-∞} | | |
| Mild impairment/control | 98.0 | 72.4-132.6 |
| Moderate impairment/control | 90.5 | 66.9-122.5 |
| Severe impairment^{†}/control | 95.8 | 69.3-132.6 |

With the exception of Cₘₐₓ in participants with severe impairment, the ratios indicated that participants with hepatic impairment had PK parameter values that were within approximately 90% of those for healthy matched participants. The mean recovery of unchanged vilazodone in urine over 96 hours, investigated in participants with mild-moderate hepatic impairment, was ≦1.3% of the dose, and similar between healthy participants and those with mild and moderate hepatic impairment. Table 12 shows pharmacokinetic parameters in subjects with hepatic impairment.

TABLE 12
Vilazodone Pharmacokinetic Parameters in Participants with Hepatic Impairment

| | Mild | | Moderate | | Severe | |
|---|---|---|---|---|---|---|
| | Impairment | Control | Impairment | Control | Impairment | Control |
| | n = 8 | n = 8 | n = 8 | n = 8 | n = 4 | n = 8 |
| Parameter* | | | | | | |
| Cₘₐₓ, ng/mL | 44.1 ± 16.5 | 43.1 ± 13.1 | 34.2 ± 8.2 | 35.8 ± 10.7 | 25.3 ± 3.8 | 35.0 ± 11.8 |
| Min-Max | 14.3-69.8 | 28.2-63.1 | 22.4-45.3 | 17.9-48.7 | 22.8-30.8 | 21.6-53.9 |
| CV | 37.4 | 30.3 | 23.9 | 29.9 | 14.8 | 33.9 |
| Tₘₐₓ, h | 4.6 ± 1.6 | 5.0 ± 1.5 | 4.1 ± 1.8 | 4.9 ± 0.84 | 4.8 ± 2.2 | 4.2 ± 1.3 |
| Median | 4.50 | 5.00 | 4.00 | 5.00 | 4.00 | 4.50 |
| Min-Max | 3.0-8.0 | 3.0-8.0 | 2.0-8.0 | 4.0-6.0 | 3.0-8.0 | 2.0-6.0 |
| CV | 34.6 | 30.2 | 43.8 | 17.1 | 46.7 | 30.2 |
| AUC₀₋ₜ, | 984 ± 505 | 898 ± 168 | 683 ± 190 | 768 ± 229 | 642 ± 96.9 | 694 ± 262 |
| ng · h/mL | | | | | | |
| Min-Max | 280-1940 | 665-1240 | 421-938 | 420-1110 | 550-772 | 438-1210 |
| CV | 51.3 | 18.7 | 27.9 | 29.9 | 15.1 | 37.7 |
| AUC_{0-∞}, | 1030 ± 506 | 945 ± 169 | 727 ± 196 | 809 ± 231 | 670 ± 94.8 | 731 ± 267 |
| ng · h/mL | | | | | | |
| Min-Max | 305-1980 | 714-1290 | 471-1020 | 448-1160 | 576-789 | 460-1260 |
| CV | 49.2 | 17.9 | 26.9 | 28.6 | 14.2 | 36.5 |
| t_{1/2}, h | 30.6 ± 8.2 | 25.9 ± 5.9 | 29.4 ± 11.4 | 25.4 ± 5.5 | 29.8 ± 4.8 | 30.1 ± 13.8 |
| Min-Max | 19.5-40.8 | 18.2-36.6 | 15.5-53.5 | 17.3-32.4 | 23.0-33.9 | 17.8-60.6 |

In the severe hepatic impairment study, concentrations of the inactive metabolite M17 were also assessed. The mean (±SD) AUC_{0-∞} of M17 was approximately 42% lower in participants with severe impairment (who did not vomit) than in normal controls (29.87±8.15 vs 51.44±21.42 ng·h/mL, respectively). The mean (±SD) pharmacokinetic parameter values of M17 in participants with severe hepatic impairment and normal controls were similar for Cₘₐₓ (1.64±0.56 vs 1.95±0.56 ng/mL, respectively), Tₘₐₓ (4.25±1.26 vs 6.00±4.14 h; median: 4.00 vs 5.00), and t_{1/2} (17.75±9.57 vs 17.35±4.25 h), suggesting that no additional accumulation of M17 following repeated dosing of vilazodone is expected in patients with severe hepatic impairment. The ratio of M17 to vilazodone (AUC_{0-∞}) was also similar between groups (0.05 vs 0.07), suggesting that vilazodone metabolism is similar in both groups.

Vilazodone was extensively bound to plasma proteins in all participants, with mean percent free fractions of 1.0-1.4% in healthy controls, 1.6% in participants with mild or moderate hepatic impairment, 2.13% in participants with severe hepatic impairment who did not vomit, and 2.24% in all participants with severe hepatic impairment. Mean unbound drug clearance (CLu/F) was also decreased (33% lower, vs matched controls) in the severe impairment group

### Safety

A listing of AEs that were reported by more than 2 participants (>5%) across both studies was constructed. AEs were roughly equivalent between participants with hepatic impairment and their matched controls, with the exception of diarrhea (10 vs 5 participants, respectively) and vomiting (4 vs 0, respectively); all 4 participants who vomited had severe hepatic impairment. All AEs were mild, except for 3 that were rated as moderate severity: 1 episode of back pain in the moderate hepatic impairment group, and 1 episode each of diarrhea and influenza in the healthy control group. No deaths, serious AEs, or discontinuations due to an AE occurred in this study.

No clinically significant changes in laboratory values, vital signs, or physical examination parameters were noted in any individual or any group from either study. None of the participants enrolled in the Severe Study reported suicidal ideation or behavior, as assessed by the C-SSRS, at any assessment (screening, day −1, or day 4). Changes in mean ECG parameters were generally small between screening and end of study, with a slight decrease of 14.9 msec in QT interval and an increase of 8.7 msec in QTcB interval (when corrected for heart rate using the Bazett formula) in the group with severe hepatic impairment; no clinically significant changes in ECG parameters were noted in the Mild-Moderate Study.

Due to the extensive hepatic metabolism of vilazodone, the current studies were performed to determine whether its pharmacokinetic profile is altered, and dose adjustments necessary, in patients with mild, moderate, or severe hepatic impairment. In these studies, all plasma pharmacokinetic parameter values in participants with mild and moderate hepatic impairment were similar to those in healthy controls, with mean values for Cₘₐₓ and AUC being within approximately 90% of the values observed in healthy matched participants.

In participants with severe hepatic impairment, mean Cₘₐₓ value of vilazodone was lower by approximately 25% compared with healthy participants, but values for AUC, Tₘₐₓ and t_{1/2} were similar between groups. Consequently, drug accumulation with repeated dosing is likely to be similar between patients with severe hepatic impairment and those with normal hepatic function. In addition, although the mean AUC_{0-∞} of the primary metabolite M17 was approximately 42% lower in participants with severe hepatic impairment than in normal controls, the mean values for Cₘₐₓ, Tₘₐₓ, and t_{1/2} were similar, suggesting that no additional accumulation of M17 is expected following repeated dosing of vilazodone in patients with severe hepatic impairment. Overall, the data from these two studies suggest that there are no clinically significant effects of mild, moderate, or severe hepatic impairment on vilazodone pharmacokinetics, and the differences observed between the populations are unlikely to require dosage adjustments Despite the fact that hepatic metabolism plays a major role in the clearance of vilazodone, its pharmacokinetics are not notably affected by hepatic impairment. This may be due to the large number of multiple metabolic pathways that can potentially be utilized during the elimination of vilazodone.

It should be noted that the recommended dosing scheme for vilazodone involves starting with 10 mg/day for 1 week, titrating upward to 20 mg/day at the beginning of week 2, and then 40 mg/day at the beginning of Week 3, such that clinically treated patients do not consume a 20 mg dose until after 7 days of 10 mg dosing. This gradual up-titration of vilazodone may improve tolerability in patients with severe hepatic impairment.

Overall, the data from these two studies suggest that there are no clinically significant effects of mild, moderate, or severe hepatic impairment on vilazodone phamiacokinetics, and the differences observed between the populations do not require dosage adjustments.

### Example 7

### Effect of Carbamazepine XR on Vilazodone Pharmacokinetics

An open-label, multiple-dose, single-sequence study to evaluate the effect of steady-state carbamazepine XR, 400 mg once daily, a CYP3A4 substrate and inducer, on the pharmacokinetics of steady-state vilazodone 40 mg tablets once daily, with hardness of more than 10 kp and less than 14 kp, preferably about 12 kp as shown in Table 3 (No Tablet Breakage). Vilazodone was co-administered with carbamazepine XR (extended release formulation). Carbamazepine is an antiepileptic drug approved for the treatment of psychomotor and grand mal seizures is metabolized by CYP3A4, and is a CYP3A4 inducer.

All subjects were healthy, nonsmoking male or female volunteers who were 18-45 (Study 2) years of age. Inclusion and exclusion criteria were typical of the criteria used in pharmacokinetic studies in healthy volunteers as known in the art. Inclusion criteria used included agreement to use an effective measure of contraception, body mass index (BMI)>18 and ≦30 kg/m² (BMI=18 allowed), normal or clinically non-significant results on physical examination, medical history, serum chemistry, hematology, and urinalysis; negative hepatitis B antigen and C antibody tests, and negative HIV test at screening. Exclusion criteria included clinically significant electrocardiogram results or QT prolongation, clinically significant disease state, history of alcohol abuse, ingestion of food, drink, or drug that may interfere with CYP enzyme activity (e.g., cruciferous vegetables, caffeine, alcohol), known sensitivity to study drug or other drugs from the same class, and use of prohibited concomitant prescription or over-the counter medications.

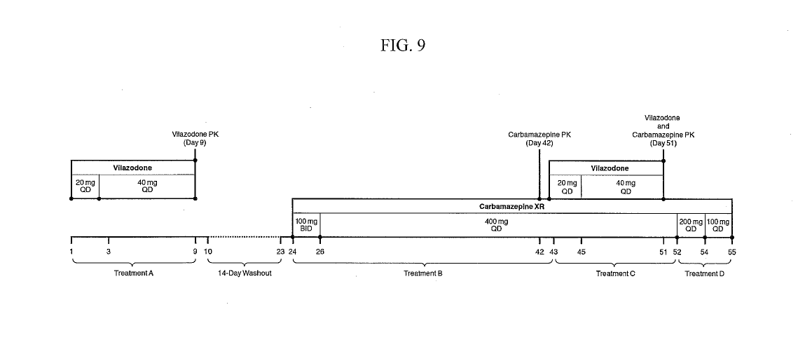

All volunteers received 4 treatments (A, B, C, and D) in the same, fixed sequence: Treatment A consisted of vilazodone alone on Days 1-9 and was followed by a 14-day washout, Treatment B was carbamazepine XR alone for 19 days (Days 24-42), Treatment C was vilazodone plus carbamazepine XR for 9 days (Days 43-51), and Treatment D was carbamazepine alone for 3 days (Days 52-54). Study design is shown in FIG. 9 of the drawings. Once-daily doses of study drug were administered in the morning after breakfast; twice-daily doses were administered in the morning after breakfast and 12 hours later in the evening after dinner Study drug was administered within 30 minutes after the end of the corresponding meal.

Blood samples to determine plasma concentrations of vilazodone, M17 (the metabolite of vilazodone), carbamazepine, and carbamazepine-10,11-epoxide (one of the pharmacologically active metabolites of carbamazepine) were collected into tubes pretreated with K₂EDTA. For vilazodone and M17 analyses, blood samples (approximately 6 mL) were collected at pre-dose on Days 1, 7, 8, 49, and 50. On Days 9 and 51, blood samples were collected at predose and at 0.5, 1, 2, 3, 4, 5, 6, 8, 10, 12, 16 and 24 hours post-dose. For the analyses of carbamazepine and carbamazepine-10,11-epoxide, blood samples were collected at pre-dose on Days 24, 40, 41, 49 and 50. On Days 42 and 51, blood samples were collected at pre-dose and at 0.5, 1, 2, 3, 4, 5, 6, 8, 10, 12, 16 and 24 hours post-dose.

Blood samples were centrifuged at no less than 2500 g for 10 minutes at 4° C. within 30 minutes of the sample draw. Plasma was harvested and transferred to polypropylene tubes and flash frozen in a dry ice bath with isopropyl alcohol or methanol. Samples for vilazodone analyses were stored at approximately −70° C. and analyzed. Samples for carbamazepine analyses were stored at −20° C. and analyzed.

Plasma samples were analyzed for vilazodone, M17, carbamazepine and/or carbamazepine-10,11-epoxide concentrations using liquid chromatography coupled with tandem mass spectrometry (LC/MS/MS). For vilazodone and M17, analytes of interest were detected by electrospray ionization mass spectrometry in the negative ion multiple reaction monitoring mode. The assay was linear in the concentration range of 0.5 to 250 ng/mL for vilazodone and 0.25 to 125 ng/mL for M17.

Based on 50 ìL of human plasma with K₂EDTA as the anticoagulant, the lower limit of quantification (LLOQ) was 0.5 ng/mL for vilazodone and 0.25 ng/mL for M17. For carbamazepine and carbamazepine-10,11-epoxide, a calibration curve was obtained from calibration standards using Analyst software (version 1.4.2), and sample concentrations were calculated from the calibration curve. Based on 50 ìL of human plasma with K₂EDTA as the anticoagulant, the LLOQ was 0.1 μg/mL for carbamazepine and 0.025 μg/mL for carbamazepine-10,11-epoxide.

### Pharmacokinetic Parameters and Statistical Analysis

The pharmacokinetic parameters for vilazodone and M17 were AUC_{0-ô,SS}, area under the plasma concentration versus time curve from time 0 to the end of the dosing interval, ô, at steady state, C_{max,SS}, maximum plasma concentration at steady state, T_{max,SS}, time to maximum plasma concentration at steady state, C_{min,SS}, minimum plasma concentration at steady state, C_{av,SS}, average plasma concentration at steady state, M17/vilazodone AUC ratio, fluctuation (C_{max,SS}−C_{min,SS})/C_{av,SS}), and swing (C_{max,SS}−C_{min,SS})/C_{min,SS}) for Treatment A (vilazodone alone) and Treatment C (vilazodone co-administered with carbamazepine). The pharmacokinetic parameters for carbamazepine and carbamazepine-10,11-epoxide were AUC_{0-ô,SS}, C_{max,SS}, T_{max,SS}, C_{min,SS}, C_{av,SS}, carbamazepine-10,11-epoxide/carbamazepine AUC ratios, fluctuation, and swing for Treatment B (carbamazepine alone) and Treatment C (carbamazepine co-administered with vilazodone). All volunteers who had evaluable pharmacokinetic parameters for both treatments in each comparison were included in the pharmacokinetic analysis, except for those volunteers who experienced emesis during the pharmacokinetic sample collection interval.

Pharmacokinetic parameters for each volunteer for vilazodone, M17, carbamazepine, and carbamazepine-10,11-epoxide analytes were analyzed by non-compartmental methods using Phoenix WinNonlin version 6.1. Pharmacokinetic parameters were compared using a linear mixed-effects model with treatment as fixed effect and subject as a random effect; statistical inference was based on log-transformed values for the metabolite ratio, C_{max,SS}, and AUC_{0-τ,SS} parameters of vilazodone and carbamazepine. Two-sided 90% CIs were constructed for the ratio of geometric means of C_{max,SS} and AUC_{0,τ,SS} of vilazodone between Treatment C and Treatment A, and of carbamazepine between Treatment C and Treatment B. No statistically significant pharmacokinetic interaction between vilazodone and carbamazepine was to be concluded if the corresponding 90% confidence intervals for the C_{max,SS} and AUC_{0-τ,SS} parameters were within the range of 80% to 125%.

Safety assessments included adverse events (AEs), clinical laboratory evaluations, vital sign assessments, 12-lead ECGs, and physical examinations. Concomitant medication checks also were conducted. Suicidal ideation and behavior was assessed with the Columbia-Suicide Severity Rating Scale (C-SSRS). For all safety assessments, the last assessment before the first dose of study drug was used as the baseline value. Safety analyses included all volunteers who received at least 1 dose of study drug.

A total of 30 volunteers were enrolled, and 23 completed the study. Reasons for discontinuation were AEs (n=3), withdrawal of consent (n=3), and lost to follow-up (n=1). The majority of volunteers were white (87%) and male (63%), and mean (SD) age and BMI were 29.3 (6.6) years and 25.4 (3.6) kg/m², respectively.

### Pharmacokinetics

Mean trough plasma concentrations of vilazodone on Days 7, 8 and 9 for Treatment A (36.00 ng/mL, 45.11 ng/mL, and 45.34 ng/mL, respectively) and Days 49, 50 and 51 for Treatment C (20.92 ng/mL, 21.90 ng/mL, and 21.39 ng/mL, respectively) indicated that steady-state levels were achieved by Day 8 for Treatment A and Day 50 for Treatment C.

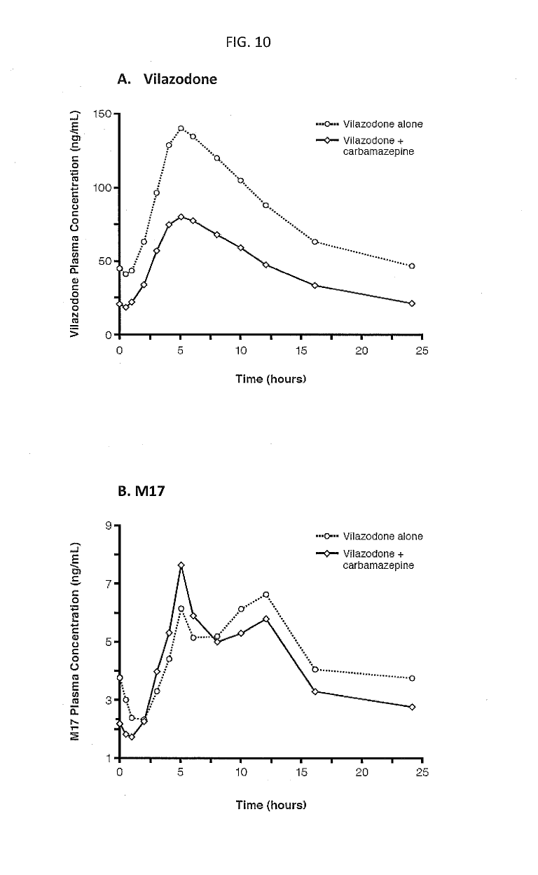

The mean plasma concentration-time profiles for vilazodone and M17 following multiple-dose vilazodone alone (Treatment A, Day 9) and with carbamazepine XR at steady state (Treatment C, Day 51) are presented in FIG. 10, and corresponding pharmacokinetic parameters for vilazodone are shown in Table 13. AUC_{0-τ,ss} and C_{max,ss} of vilazodone were decreased by 44.5% and 40.8%, respectively, following co-administration of vilazodone with carbamazepine verses vilazodone alone.

Vilazodone C_{min,ss} and C_{avg,ss} were decreased approximately 50% following co-administration verses vilazodone alone, while median t_{max,ss} appeared to be unchanged. The 90% CIs for the geometric means ratios of AUC_{0-τ,ss} and C_{max,ss} were not within the range of 80% to 125%, indicating that there was a statistically significant effect of carbamazepine XR co-administration on the pharmacokinetics of vilazodone.

Mean AUC_{0-τ,ss} and C_{max,ss} of the vilazodone metabolite M17 were similar when vilazodone was administered alone and upon co-administration with carbamazepine XR (AUC_{0-τ,ss} of 110.4 ng·h/mL and 100.1 ng·h/mL, respectively, and C_{max,ss} of 7.2 ng/mL and 8.0 ng/mL, respectively) as shown in Table 12. Median t_{max,ss} appeared to be shortened to 5 h from 10 h for M17 following co-administration of vilazodone with carbamazepine XR vs vilazodone alone. Mean fluctuation and swing of M17 were increased by approximately 70% and 96%, respectively, following co-administration of vilazodone and carbamazepine XR vs vilazodone alone.

TABLE 13
Pharmacokinetic Parameters of Vilazodone and Metabolite M17 Following the
Multiple-dose Administration of 40-mg Vilazodone Alone and With Co-administration
of 400-mg Carbamazepine at Steady-state (PK Population, N = 23)

| | | | Ratio of Geometric | |
|---|---|---|---|---|
| PK Parameters | VLZ alone | VLZ + CBZ | Means % (VLZ + | |
| of Vilazodone | Mean (SD) | Mean (SD) | CBZ vs VLZ alone | 90% CI |
| AUC_{0-τ, ss}, ng · h/mL | 1,976.0 (516.3) | 1,092.0 (272.2) | 55.5 | 53.1-58.0 |
| C_{max, ss}, ng/mL | 147.9 (38.0) | 88.2 (26.0) | 59.2 | 55.4-63.3 |
| C_{min, ss}, ng/mL | 47.5 (18.8) | 22.4 (8.8) | — | — |
| C_{avg, ss}, ng/mL | 82.3 (21.5) | 45.5 (11.3) | — | — |
| tₘₐₓ^{a}, h | 5 (4-12) | 5 (3-10) | — | — |

| PK Parameters of | VLZ Alone | VLZ + CBZ |
|---|---|---|
| M17 | Mean (SD) | Mean (SD) |
| AUC_{0-τ, ss}, ng · h/mL | 110.4 (29.13) | 110.1 (21.5) |
| C_{max, ss}, ng/mL | 7.2 (2.1) | 8.0 (1.9) |
| C_{avg, ss}, ng/mL | 4.6 (1.2) | 4.2 (0.9) |
| tₘₐₓ^{a}, h | 10 (5-12) | 5 (5-12) |

^{a}Median (min-max).
VLZ = vilazodone; CBZ = carbamazepine; VLZ + CBZ = co-administration of vilazodone and carbamazepine.

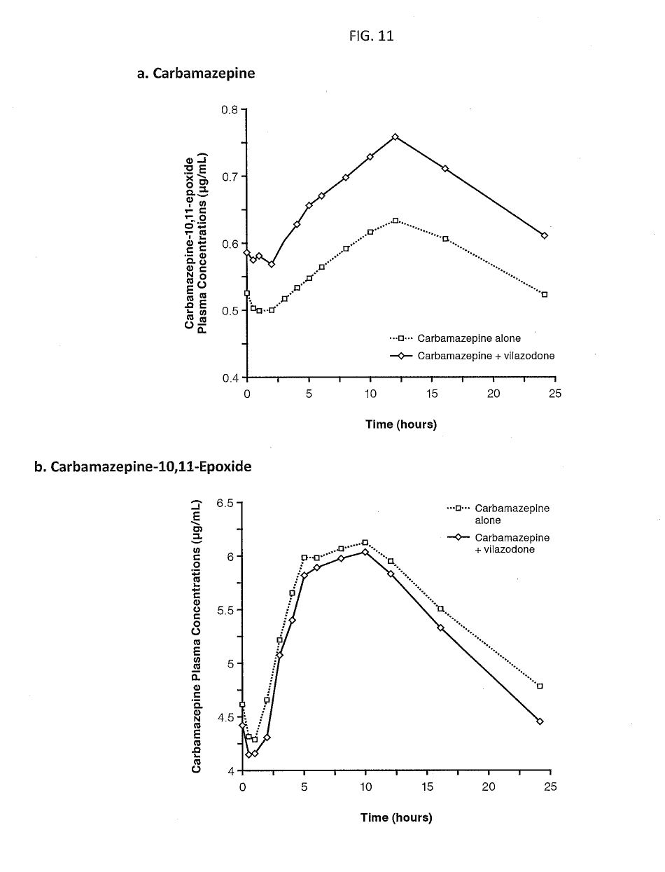

Mean trough plasma concentrations of carbamazepine on Days 40, 41 and 42 for Treatment B (5.11 μg/mL, 4.70 μg/mL, and 4.61 μg/mL, respectively) and Days 49, 50 and 51 for Treatment C (4.37 μg/mL, 4.50 μg/mL, and 4.44 μg/mL, respectively) indicated that steady-state levels were achieved by Day 41 for Treatment B and Day 50 for Treatment C. The mean plasma concentration-time profiles for carbamazepine and carbamazepine-10,11-epoxide following multiple-dose carbamazepine XR alone (Treatment B, Day 42) and with vilazodone at steady state (Treatment C, Day 51) are presented in FIG. 11, and corresponding pharmacokinetic parameters for carbamazepine are shown in Table 14. For carbamazepine, co-administration with steady-state vilazodone slightly increased mean fluctuation (˜20%) and swing (˜25%); otherwise, co-administration did not seem to affect carbamazepine pharmacokinetic parameters as shown in Table 14. The 90% CIs for the geometric means ratios of AUC_{0-τ,ss} and C_{max,ss} were within the range of 80% to 125%, indicating that there was not a statistically significant effect of vilazodone co-administration on the pharmacokinetics of carbamazepine XR.

TABLE 14
Pharmacokinetic Parameters Of Carbamazepine and metabolite carbamazepine-10,11-
epoxide Following the Multiple-Dose Administration of 400-mg Carbamazepine Alone
and With Co-administration of 40-mg Vilazodone (PK Population, N = 23)

| | | | Ratio of Geometric | |
|---|---|---|---|---|
| PK Parameters | CBZ alone | CBZ + VLZ | Means % (CBZ + | |
| of Carbamazepine | Mean (SD) | Mean (SD) | VLZ vs CBZ alone) | 90% CI |
| AUC_{0-τ, ss}, μg · h/mL | 131.3 (16.5) | 127.2 (18.8) | 96.6 | 92.7-100.6 |
| C_{max, ss}, μg/mL | 6.4 (0.7) | 6.4 (0.9) | 99.2 | 95.0-103.6 |
| C_{avg, ss}, μg/mL | 5.5 (0.7) | 5.3 (0.8) | — | — |
| tₘₐₓ^{a}, h | 10 (4-16) | 10, (5-12) | — | — |

| PK Parameters of | | |
|---|---|---|
| Carbama/epine- | CBZ alone | CBZ + VLZ |
| 10,11-epoxide | Mean (SD) | Mean (SD) |
| AUC_{0-τ, ss}, μg · h/mL | 13.7 (3.7) | 16.1 (5.1) |
| C_{max, ss}, μg/mL | 0.7 (0.2) | 0.8 (0.2) |
| C_{avg, ss}, μg/mL | 0.6 (0.2) | 0.7 (0.2) |
| tₘₐₓ^{a}, h | 12 (6-16) | 12 (5-16) |

^{a}Median (min-max).
CBZ = carbamazepine; VLZ = vilazodone; CBZ + VLZ = co-administration of vilazodone and carbamazepine.

For carbamazepine-10,11-epoxide, a small increase (˜18%) in mean AUC_{0-τ,ss} (16.1 μg˜h/mL vs 13.7 μg·h/mL) and C_{max,ss} (0.77 μg/mL vs 0.65 μg/mL) was observed when carbamazepine XR was co-administered with vilazodone vs carbamazepine XR alone as shown in Table 14. Median t_{max,ss} for carbamazepine-10,11-epoxide appeared to be unaffected (12.0 h), and mean C_{min,ss}, C_{avg,ss}, swing, and fluctuation were slightly increased by ˜15%, ˜17%, 15%, and 14%, respectively, during co-administration vs carbamazepine XR alone.

### Safety and Tolerability

A total of 29 participants reported at least 1 AE. About 97% of all AEs were mild in intensity, and approximately 84% were considered to be related to the study drugs. The 3 subjects who discontinued due to AEs (full body hives, emotional lability, and lower abdominal pain in 1 subject each) did so during or after completion of Treatment B. All AEs resolved after discontinuing study drug. No participant had CSSR-based suicidal behavior during the study; however, 1 subject with an undisclosed history of depression and increased suicidal thoughts with administration of multiple antidepressants reported suicidal ideation and was prematurely discontinued from the study (AE of emotional lability). There were no serious AEs or deaths during study. Small changes from baseline were observed for laboratory parameters, vital signs, and 12-lead ECGs, but none were considered clinically significant.

The co-administration of vilazodone and carbamazepine XR, a CYP3A4 inducer, decreased mean steady-state vilazodone exposure by about 45%. The 90% CIs for the ratio (vilazodone plus carbamazepine XR vs vilazodone alone) of geometric means for AUC and Cₘₐₓ were not within the range of 80% to 125%, indicating a statistically significant effect of carbamazepine on the pharmacokinetics of vilazodone. These results suggest that higher doses of vilazodone should be considered when vilazodone is given in combination with strong CYP3A4 inducers.

The effect on the vilazodone metabolite M17 was highly variable with carbamazepine XR co-administration, describing the inter-individual variability in the inhibition and induction of CYP enzymes. Co-administration of carbamazepine XR with vilazodone did not appear to appreciably affect the steady-state pharmacokinetic parameters of carbamazepine, as the 90% CIs for the ratio (vilazodone plus carbamazepine XR vs carbamazepine XR alone) of geometric means for AUC and Cₘₐₓ were within the range of 80% to 125%. Co-administration of carbamazepine XR with vilazodone also had minimal effect on the pharmacokinetic parameters of the carbamazepine-10,11-epoxide metabolite. This outcome is consistent with in vitro studies which demonstrate that vilazodone is not an inhibitor or inducer of CYP3A4. The pharmacokinetic parameters of vilazodone observed in were similar to previously reported values.

Co-administration of vilazodone and carbamazepine XR at steady state was also generally safe and well tolerated, and gastrointestinal AEs (i.e., nausea, vomiting, and diarrhea) were among the most commonly reported AEs, consistent with the reported safety profile of vilazodone. The AE results reported during open-label administration should be regarded with caution due to the limitations of the open-label design. There were slight changes from baseline in laboratory results, vital signs, and 12-lead ECG parameters, though they were not considered clinically relevant.

Co-administration of vilazodone and carbamazepine XR (a CYP3A4 inducer) at steady state resulted in a significant decrease in vilazodone exposure (45%). No significant effect of vilazodone on the pharmacokinetics of carbamazepine (a CYP3A4 substrate) was observed. Administration of vilazodone with carbamazepine was generally well tolerated in healthy participants.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. An immediate release oral dosage form comprising therapeutically effective amount of vilazodone or a salt thereof and at least one excipient, wherein the dosage form comprises 10 to 40 mg of vilazodone or a salt thereof, and is compressed in a tablet formulation, and wherein
the tablet comprising 40 mg vilazodone or a salt thereof has a hardness of more than 9 kp and less than or equal to 14 kp;
the tablet comprising 20 mg vilazodone or a salt thereof has a hardness of more than 8 kp and less than or equal to 12 kp; and
the tablet comprising 10 mg vilazodone or a salt thereof has a hardness of more than 6 kp and less than or equal to 9 kp.

2. The oral dosage form according to claim 1, wherein the oral dosage form is of thickness of about 0.095-0.195″ and friability is not more than about 1.0%.

3. The oral dosage form according to claim 1, wherein the tablet has a disintegration time of not more than about 2 minutes and a compression force of about 4 kN to 18 kN.

4. The oral dosage form according to claim 1, wherein the excipient comprises one or more diluents, disintegration aids, glidants, lubricants, coloring agents, and opacifying agents.

5. The oral dosage form according to claim 1, wherein the vilazodone or a salt thereof is present in amounts ranging from about 0.05% w/w to about 50% w/w.

6. The oral dosage form according to claim 4, wherein the diluent is selected from the group consisting of lactose, sucrose, glucose, dextrose, microcrystalline cellulose, dibasic calcium phosphate, calcium sulphate, mannitol, erythritol, lactilol, maltitol, xylitol, sorbitol, starch, and mixtures thereof.

7. The oral dosage form according to claim 6, wherein the diluent is present in amounts ranging from about 20% w/w to about 80% w/w.

8. The oral dosage form according to claim 4, wherein the glidant is selected from the group consisting of water soluble excipient, hydrophilic polymers, silicon dioxide, colloidal silicon dioxide, microcrystalline cellulose, and combinations thereof.

9. The oral dosage form according to claim 8, wherein the glidant is present in amounts ranging from about 0.05% w/w to about 5% w/w.

10. The oral dosage form according to claim 4, wherein the disintegration aid is selected from the group consisting of ion exchange resin, hydroxypropylcellulose, crospovidone, croscarmellose sodium, starches, pectins, alginates, surfactants, microcrystalline cellulose, sodium starch glycolate, and combinations thereof.

11. The oral dosage form according to claim 10, wherein the disintegration aid is present in amounts ranging from about 5% w/w to about 30% w/w.

12. The oral dosage form according to claim 4, wherein the lubricant is selected from the group consisting of magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, zinc stearate, and combinations thereof.

13. The oral dosage form according to claim 12, wherein the lubricant is present in amounts ranging from about 0.3% w/w to about 10% w/w.

14. An immediate release oral dosage form according to claim 1, wherein the dosage form comprises 20 mg vilazodone or a salt thereof and produces in subjects with severe hepatic impairment an effect comprising at least one of:
a mean Cₘₐₓ of about 22 ng/ml or more;
a mean Tₘₐₓ of about 3 hours or more; or
a mean AUC_{0-∞} of more than about 550 ng h/ml.

15. The immediate release oral dosage form of claim 14, wherein the mean Cₘₐₓ in subjects with severe hepatic impairment is about 25% lower than mean Cₘₐₓ of subjects without severe hepatic impairment.

16. The immediate release oral dosage form of claim 14, comprising pharmacokinetic profile as shown in FIG. 8.

17. The immediate release oral dosage form of claim 14, wherein the oral dosage form produces in subjects with moderate hepatic impairment an effect comprising at least one of:
a mean Cₘₐₓ of about 22 ng/ml or more;
a mean Tₘₐₓ of about 2 hours or more; or
a mean AUC_{0-∞} of more than about 450 ng h/ml.

18. The immediate release oral dosage form of claim 17, comprising pharmacokinetic profile as shown in FIG. 7.

19. The immediate release oral dosage form of claim 14, wherein the oral dosage form produces in subjects with mild hepatic impairment an effect comprising at least one of
a mean Cₘₐₓ of about 14 ng/ml or more;
a mean Tₘₐₓ of about 3 hours or more; or
a mean AUC_{0-∞} of more than about 300 ng h/ml.

20. The immediate release oral dosage form of claim 19, comprising pharmacokinetic profile as shown in FIG. 7.

21. The immediate release oral dosage form of 14, wherein the oral dosage form produces in the subjects a concentration of metabolite M17 of vilazodone or a salt thereof, having a structure 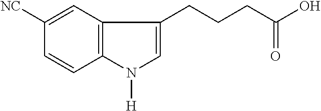 [SMILES: N#Cc1ccc2[nH]cc(CCCC(=O)O)c2c1]
comprising at least one of:
a mean Cₘₐₓ of about 1 ng/ml or more;
a mean Tₘₐₓ of about 3 hours or more; or
a mean AUC_{0-∞} of more than about 20 ng h/ml.

22. The immediate release oral dosage form of claim 21, wherein the concentration of metabolite M17 of vilazodone or a salt thereof is at least 42% lower in subjects with severe impairment than the concentration of M17 in subjects without severe hepatic impairment.

23. An immediate release oral dosage form according to claim 1, wherein the dosage form comprises 40 mg of vilazodone or a salt thereof when administered to healthy subjects produces an in vivo plasma profile of metabolite M17 of vilazodone or a salt thereof, comprising at least one of:
a mean Cₘₐₓ of about 5 ng/ml or more;
a mean Tₘₐₓ of about 5 hours or more; or
a mean AUC₀₋₂₄ of more than about 80 ng h/ml.

24. An immediate release oral dosage form according to claim 1, wherein the dosage form of 40 mg of vilazodone or a salt thereof produces an in vivo plasma profile of vilazodone or a salt thereof, comprising at least one of:
a mean Tₘₐₓ of about 4 hours or more;
a mean Cₘₐₓ of less than about 180 ng/ml; or
a mean AUC₀₋₂₄ of less than about 1800 ng h/ml.

25. A method of treating a subject with severe hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising administering to said subject the oral dosage form according to claim 14.

26. A method of treating a subject with moderate hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising administering to said subject the oral dosage form according to claim 17.

27. A method of treating a subject with mild hepatic impairment suffering from a depressive disorder, an anxiety disorder, a bipolar disorder, mania, dementia, a substance-related disorder, a sexual dysfunction, an eating disorder, obesity, fibromyalgia, a sleeping disorder, a psychiatric disorder, cerebral infarct, tension, side-effects in the treatment of hypertension, a cerebral disorder, chronic pain, acromegaly, hypogonadism, secondary amenorrhea, premenstrual syndrome, undesired puerperal lactation, or combinations thereof, comprising administering to said subject the oral dosage form according to claim 19.
